(19) United States
(12) Patent Application Publication
(10) Pub. No.: US 2025/0152504 A1
(43) Pub. Date: May 15, 2025

(54) INTRAUTERINE SYSTEMS COMPRISING ANTI-INFECTIVES FOR STEADY-STATE ACCUMULATION TO DISTAL EXTRAUTERINE TISSUE

(71) Applicants: University of Washington, Seattle, WA (US); Oregon Health & Science University (OHSU), Portland, OR (US)

(72) Inventors: Hannah VanBenschoten, Seattle, WA (US); Kim Woodrow, Seattle, WA (US); Ian Suydam, Seattle, WA (US); Jeffrey Jensen, Portland, OR (US); Bob Katz, Seattle, WA (US)

(73) Assignees: University of Washington, Seattle, WA (US); Oregon Health & Science University (OHSU), Portland, OR (US)

(21) Appl. No.: 18/941,995

(22) Filed: Nov. 8, 2024

Related U.S. Application Data
(60) Provisional application No. 63/597,840, filed on Nov. 10, 2023.

(51) Int. Cl. A61K 9/00; A61K 31/439; A61K 31/4439; A61K 31/505; A61K 31/513; A61K 31/522; A61K 31/65; A61K 47/34

(52) U.S. Cl. CPC A61K 9/0039; A61K 31/439; A61K 31/4439; A61K 31/505; A61K 31/513; A61K 31/522; A61K 31/65; A61K 47/34

(57) ABSTRACT

An intrauterine system configured to be retained in a uterus of a patient, the intrauterine system including a delivery system configured to deliver one or more active compounds, where the one or more active compounds have non-systemic extrauterine biological targets. Further, a method of using the intrauterine system, including retaining the intrauterine system in a uterus, and delivering the one or more active compounds to non-systemic extrauterine biological targets in the lower female reproductive tract (LFRT) or the rectum.

### CROSS-REFERENCE TO RELATED APPLICATION

This application claims the benefit of U.S. Provisional Application No. 63/597,840 filed Nov. 10, 2023, the entire disclosure of which is hereby incorporated by reference.

### STATEMENT OF GOVERNMENT LICENSE RIGHTS

This invention was made with government support under Grant No. R01AI50325-02, awarded by the National Institutes of Health. The government has certain rights in the invention.

### BACKGROUND

Women are disproportionately burdened with sexual, reproductive, and gynecological conditions across their lifespan that can significantly reduce their quality of life. In particular, protection from and treatment of lower female genital tract infections is ranked third in terms of global burden of disease, contributing to approximately 14 million disability-adjusted life years (DALYs), with more than 1 million deaths annually due to sexually transmitted infections (STIs) including human immunodeficiency virus (HIV). As such, there is a significant clinical need for safe and effective prophylactic and therapeutic strategies that are adherence-independent, long-acting, and highly reversible that deliver biologically active compounds to anatomical sites of infection or recurrent disease.

Prevention and treatment of sexually transmitted infections (STIs) that are acquired vaginally necessitates pharmacologic intervention at the site of pathogen infection and replication, thus anti-infective drug must be present in the female reproductive-genital tract (FRGT) and anus/rectum to prevent or treat local infections and symptomatic occurrences. As used herein, the female reproductive-genital tract (FRGT) includes the upper (cervix, uterus/endometrium, fallopian tubes) and lower (vaginal, vulvar, vestibule and vestibular glands, and vulva) organs. Direct drug delivery to these anatomical sites is crucial to interrupt the infection cycle, mitigate disease progression, and prevent recurrent infections. By delivering therapeutics or prophylactics to the exact site of infection, pharmacologic efficacy may be enhanced while minimizing pharmacokinetic challenges such as systemic side effects, hepatic first-pass metabolism, and limited drug partitioning to mucosal tissue. The current landscape of prevention and treatment technologies for infections of the FRGT and anus/rectum primarily includes oral medications and topical formulations. Oral antibiotics and antivirals are commonly used for treating STIs, but adherence issues and the potential for systemic side effects remain challenges. Topical treatments, such as gels and intravaginal rings, offer targeted drug delivery, improving localized drug concentration and reducing systemic exposure. These approaches, while promising, require further development to optimize long-acting, adherence-independent solutions that are both acceptable and effective in preventing and treating infections.

The feasibility of local, directional transport at steady-state from the upper reproductive tract (cervix, uterus/endometrium, fallopian tubes) to the lower genital tract (vaginal, vulvar, vestibule and vestibular glands, and vulva) and anus/rectum has not previously been known or demonstrated. No pharmacokinetic data previously existed describing extrauterine (e.g., vaginal, vulvar, vestibule, secretions, anus/rectum) drug accumulation resulting from deposits or delivery of bioactive compounds that originate in or from the uterus. As a consequence, the current landscape for biomedical use of intrauterine systems is described entirely for biologically active compounds that accumulate and act locally within the endometrium and uterine lining for contraception and dysmenorrhea to generate a hostile environment for sperm (copper-IUD) and/or suppress cervical mucus, thin and atrophy the endometrium to decrease uterine contractions and menstrual bleeding, and create barriers to sperm motility and function (hormonal-IUD).

Active myometrial contractions and cervical peristalsis during the processes of menstruation and childbirth are known to transport uterine contents inferiorly through the cervical lumen, but there is no evidence of steady-state (e.g., not during menses and childbirth) uterine-to-vaginal drug transport that would premise sustained and long-acting therapeutic dosing to extrauterine targets. With the exception of these intermittent and hormone-controlled active transport processes, the uterine first-pass-effect, which is the most studied transport phenomenon of the female reproductive tract, contradicts any steady-state transport from the uterus to the lower female genital tract. The uterine-first-pass-effect describes vaginal-to-uterine transport that results in uterine accumulation of vaginally administered substances concomitant with minimum systemic absorption. The preferential transport of vaginally administered drugs to the uterus has been proposed based on evidence of: (1) high drug accumulation in endometrial tissue from vaginal but not oral or intramuscular administration, (2) low systemic plasma drug concentration from vaginal but not oral or intramuscular administration, and (3) high plasma drug concentration from oral or intramuscular administration with minimal uterine accumulation and weak endometrial pharmacodynamics. These findings have been replicated for a variety of active agents including the androgen receptor danazol, estradiol, misoprostol for medical abortion, and mifepristone for treatment of uterine leiomyomata. The physiological mechanisms that accounts for the uterine-first-pass-effect are described by four theories that explain vaginal-to-uterine transport via direct diffusion through local tissues, active uterine peristalsis, venous or lymphatic transport associated with vessels originating from the cranial part of the vagina to the uterine cervix, and finally countercurrent exchange between the utero-ovarian vein and the ipsilateral ovarian artery with the vaginal venous plexus in the upper third of the vagina and uterus. Likewise, there is only one published study that has measured local tissue pharmacokinetics from drugs administered intrauterine, which detected high local tissue concentrations within the upper female reproductive tract (endometrium, myometrium, fallopian tubes) and strongly premises local intrauterine rather than distally extrauterine in the lower genital tract (vaginal, vulvar, vestibule and vestibular glands, and vulva) and anus/rectum.

Intrauterine devices, particularly for contraception and/or management of heavy menstrual bleeding, have an extensive history of development and wealth of clinical data supporting their safety and efficacy, which offers a paradigm for developing a uterine system that also functions as a long-acting preventative or treatment for other genitourinary (including the lower FGT and the anus/rectum) indications. However, the success of contraceptive hormonal- and copper-IUDs is strongly potentiated by potent compounds that are sustained for years and act locally in the upper female reproductive tract (cervix, uterus/endometrium, fallopian tubes). Intrauterine systems (drugs, devices, and drug-device combinations) have not been designed nor have they demonstrated the ability for steady-state uterine-to-vaginal transport of biologically active compounds that would provide sustained and long-acting dosing to extrauterine targets in the lower genital tract (vaginal, vulvar, vestibule and vestibular glands, and vulva) and anus/rectum for a minimum of weeks to months.

Accordingly, there is a pressing need for more innovative drug delivery systems to enhance treatment outcomes in the FRGT and anus/rectum.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, disclosed herein is a drug delivery system configured to be retained in a uterus of a patient, and to deliver one or more active compounds, wherein the one or more active compounds have non-systemic extrauterine biological targets.

In some embodiments, the non-systemic extrauterine biological targets are in a lower female genital tract (LFGT) of the patient. In some embodiments, the non-systemic extrauterine biological targets are in an anus or a rectum of the patient. In some embodiments, the delivery system is configured to accumulate the one or more active compounds in the LFGT, the anus, or the rectum for an extended duration.

In some embodiments, the intrauterine system is configured to facilitate uterine-to-LFGT transport of the one or more active compounds, and wherein the one or more active compounds are detectable in tissue, local secretions, or a combination thereof at least 7 days from placement of the intrauterine system into a uterus. In some embodiments, the tissue is selected from cervical tissue, uterus tissue, endometrium tissue, fallopian tube tissue, vaginal tissue, vulvar tissue, vestibule tissue, vestibular glands, uterus tissue, ovarian tissue, or a combination thereof. In some embodiments, the one or more active compounds have a steady-state drug concentration of at least 0.5-25 ng/mg in the tissue. In some embodiments, the one or more active compounds have a steady-state drug concentration of at least 0.5-25 ng/mL in the local secretions.

In some embodiments, the delivery system includes a core formed from a core polymer, wherein the one or more active compounds are dispersed within the polymer core. In some embodiments, the core polymer comprises thermoplastic polyurethane (TPU). In some embodiments, the delivery system further comprises a sheath membrane surrounding the core.

In some embodiments, the one or more active compounds are configured to treat one or more conditions selected from human immunodeficiency virus (HIV), herpes, chlamydia, gonorrhea, syphilis, Herpes Simplex Virus (HSV), and bacterial vaginosis.

In some embodiments, the one or more active compounds are selected from raltegravir (RAL), maraviroc (MVC), etravirine (ETR), dapivirine (DPV), pritelivir, acyclovir, doxycycline, or a combination thereof.

In some embodiments, the one or more active compounds are configured to accumulate non-systemically in cervical tissues, vaginal tissues, vaginal vestibule tissues, rectum tissues, vaginal fluids, or a combination thereof in a concentration that is uterine-distance dependent.

In some embodiments, the one or more compounds is configured to release from the delivery system at an effective level for up to about ten years. In some embodiments, the effective level ranges from about 1 μg to about 500 μg a day.

In another aspect, disclosed herein is a intrauterine system, including at least one drug delivery system as described herein, and an intrauterine frame.

In some embodiments, the intrauterine system is further configured to prevent pregnancy.

In some embodiments, the drug delivery system is a first drug delivery system, and wherein the intrauterine system further comprises a second drug delivery system, wherein the first drug delivery system releases an active compound of the one or more active compounds and wherein the second drug delivery system releases a different active compound of the one or more active compounds.

In yet another aspect, disclosed herein is a method of using the drug delivery system as described herein, the method including retaining the drug delivery system in a uterus, and delivering the one or more active compounds to non-systemic extrauterine biological targets in the LFGT, the anus, or the rectum.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A is an example intrauterine system with an example drug delivery system, in accordance with the present technology;

FIG. 1B is another example intrauterine system with two delivery systems, in accordance with the present technology;

FIG. 1C is yet another example intrauterine system having a plurality of delivery systems, in accordance with the present technology;

FIG. 1D is yet another example intrauterine system having one or more contraceptive elements, in accordance with the present technology;

FIGS. 2A-2B is an example cross-section and perspective view of delivery systems, respectively, in accordance with the present technology;

FIGS. 3A-3B is an example cross-section and perspective view of delivery systems, respectively, in accordance with the present technology;

FIGS. 4A-4F are example process steps of preparing a delivery system, in accordance with the present technology;

FIG. 5 is a diagram of an example intrauterine device retained in a uterus, in accordance with the present technology;

FIGS. 6A-6C are graphs showing differential dapivirine release driven by polymer crystallinity and water penetration, in accordance with the present technology;

FIGS. 7A-7B are graphs of high loaded dapirivine thermoplastic polyurethane 4, in accordance with the present technology;

FIG. 8 is a graph showing 3-antiretroviral dissolution is dependent on active pharmaceutical ingredient and polymer properties, in accordance with the present technology;

FIGS. 9A-9B are graphs of thermoplastic polyurethane highly loaded with raltegravir, etravirine, and maraviroc, in accordance with the present technology;

FIGS. 10A-10B are graphs of thermoplastic polyurethane highly loaded with raltegravir and maraviroc achieving one year of release or greater, in accordance with the present technology;

FIGS. 11A-11B are graphs showing in vitro release of an example delivery system loaded with 1 wt % of dapivirine, in accordance with the present technology;

FIGS. 12A-12G are graphs illustrated dapivirine concentrations in tissue, secretions, and plasma of example intrauterine devices inserted into a baboon uteri;

FIGS. 13A-13D are graphs of tissue concentrations of raltegravir, etravirine, and maraviroc, in the lower female reproductive tract and the rectum, in accordance with the present technology;

FIG. 14 is a graph showing the concentration of anti-retroviral pharmaceutical compositions in vaginal secretions, in accordance with the present technology;

FIGS. 15A-15C are graphs showing post-use assays of residual anti-retroviral pharmaceutical compositions in removed intrauterine devices, in accordance with the present technology;

FIGS. 16A-16C are graphs showing concentrations of anti-retroviral pharmaceutical compositions in the vaginal vault, in accordance with the present technology;

FIGS. 17A-17C are graphs showing the in vitro release of pritelivir, in accordance with the present technology;

FIGS. 18A-18B are graphs showing daily doses of released acyclovir and doxycycline, respectively, in accordance with the present technology; and

FIGS. 19A-19B are graphs showing release for dapirivine in ethylene-vinyl acetate polymers, in accordance with the present technology.

### DETAILED DESCRIPTION

Described herein are intrauterine systems that provide a source and delivers steady-state therapeutically effective doses of biologically active compounds that accumulate in extrauterine target tissues and distally to the female reproductive-genital tract (FRGT) and anus/rectum via non-systemic direct uterine-to-vaginal drug transport for extended durations. As used herein, the term female reproductive genital tract is understood to mean the upper (cervix, uterus/endometrium, fallopian tubes) and lower (vaginal, vulvar, vestibule and vestibular glands, and vulva) organs.

In one aspect, disclosed herein is an intrauterine system (IUS) that functions as a long-acting preventative or treatment for female genitourinary indications. In some embodiments, the IUS is configured to prevent sexually transmitted infections or treat sexually transmitted disease for extended durations, without requirement for continuous and active end-user involvement.

In another aspect, disclosed herein is an IUS comprising a source of one or more biologically active compounds (or “one or more active compounds”) that have biological targets that are non-systemic and extrauterine in the female reproductive-genital tract (FRGT) and/or anus/rectum. In one aspect, the IUS is configured to be retained in the uterus of female mammals with a biological active compound deposit and rate-controlling polymers and structures to provide drug detection in extrauterine tissue at steady-state for extended durations.

In some embodiments, the IUS enables uterine-to-vaginal transport of the one or more active compounds from the source. In some embodiments, the one or more active compounds may be detected in at least 5 days in a uterine distance-dependent gradient. In some embodiments, the uterine distance-dependent gradient is detectable from the endometrium/uterus, cervix, FRGT, and rectal tissue and local secretions. In some embodiments, the IUS results in detection of drug concentrations in the endometrium of 100-500 ng/g, cervix of 50-500 ng/g, vagina of 10-500 ng/g, and vaginal secretions of 10 to 10⁵ ng/mL. In some embodiments, the IUS results in detection of drug concentrations in endometrium of 500-2000 ng/g, cervix of 100 ng/g, vagina of 50-250 ng/g, and vaginal secretions of 10² to >10⁴ ng/mL.

In some embodiments, the IUS results in detection of drug (also referred to herein as “biologically active compound” or “pharmaceutical composition”) concentrations distally in the vaginal vestibule and/or rectum. In some embodiments, the IUS results in detection of drug concentrations distally in the vaginal vestibule of 10-500 ng/g and rectum of 5-200 ng/g. In some embodiments, the IUS results in detection of drug concentrations distally in the vaginal vestibule of 50-1000 ng/g and rectum of 25-100 ng/g.

In some embodiments, the IUS results in low plasma drug levels concomitant with high local tissue concentration. In some embodiments, the IUS results in steady-state plasma drug levels that are at least 1000× lower than tissue concentrations. In some embodiments, the IUS results in steady-state plasma drug levels that are <200 μg/mL.

In some embodiments, the IUS has measurable residual drugs that support durable sustained release of the one or more biologically active compounds for extended durations. In some embodiments, the residual drug content indicates daily release rates that are sustained for a minimum of 7-60 days. In some embodiments, the residual drug content provides for daily release rates of 15 μg/day-100 μg/day.

In some embodiments, the IUS is configured with one or more drug delivery systems to release the one or more active compounds extrauterine. In some embodiments, the one or more delivery systems are a monolithic matrix. In other embodiments, the one or more drug delivery systems are a core-sheath reservoir. In some embodiments, the one or more drug delivery systems include a combination of monolithic matrix and core-sheath reservoir drug delivery systems. The monolithic matrix drug delivery system may include a drug (or “pharmaceutical composition”) homogenously dispersed or dissolved within a polymer matrix. In some embodiments, such drug delivery devices release the drug with first order or square root of time release kinetics in which the rate of drug release decreases over time. In such systems, drug release can be modulated by the geometry of the device, particularly the surface area to volume ratio, polymer swelling, porosity, water uptake, and active pharmaceutical ingredient (API) diffusivity through the matrix. The core-sheath reservoir may be capable of achieving zero-order release for several years given that a drug is loaded in excess, i.e., above the saturation solubility, of the core polymer. In some embodiments, release is modulated by the diffusion coefficient of drug in the sheath polymer, thickness of the rate-controlling membrane, partitioning coefficient of drug between the core and sheath, and dimensions of the device.

In some embodiments, the drug delivery system includes a polymer core, where the pharmaceutical composition (or one or more active compounds) is dispersed within the polymer core. In some embodiments, the polymer core includes thermoplastic polyurethane (TPU), ethylene vinyl acetate (EVAs), and/or biodegradable polyesters. In some embodiments, the polymer core is hydrophobic.

In some embodiments, the drug delivery system has minimum dimensions of about 3.8 mm to about 4.4 mm in width and about 30 mm to about 32 mm in height. In some embodiments, the dimensions allow passage through the cervix for placement in the uterus. In some embodiments, the drug delivery system is placed in the uterus by hysterotomy.

In some embodiments, the drug delivery system further includes a sheath membrane surrounding the polymer core. In some embodiments, the sheath membrane has a thickness between 50-500 μm. In some embodiments, the sheath membrane includes a polymer. In some embodiments, the polymer of the sheath membrane is distinct from the polymer core. In some embodiments, the polymer of the sheath membrane is hydrophobic. In some embodiments, the sheath membrane polymer includes TPU.

In some embodiments, the pharmaceutical composition is above the saturation solubility (Cs) of the polymer core and below the Cs of the sheath membrane.

In some embodiments, the drug delivery system further includes an opening. In some embodiments, a frame or retrieval filament of the IUS is configured to thread through the opening.

In some embodiments, the one or more biologically active compounds are hydrophilic (i.e., where log P<1). In some embodiments, the one or more biologically active compounds are hydrophobic (i.e., where log P>5). In some embodiments, the one or more biologically active compounds are of intermediate hydrophobicity (i.e., where 1<log P<5). In some embodiments, the one or more biologically active compounds have a water solubility ranges from >50 mg/mL to <10⁻³ mg/mL. In some embodiments, the one or more biologically active compounds are present in the delivery system at an amount above the saturation solubility of the polymer of the polymer core.

In some embodiments, the one or more active compounds (or pharmaceutical compounds) are configured to treat one or more sexually transmitted infections or diseases. In some embodiments, the one or more sexually transmitted infections or diseases are selected from human immunodeficiency virus (HIV), herpes, chlamydia, gonorrhea, syphilis, and bacterial vaginosis. In some embodiments, the biologically active compound is an anti-infective. In some embodiments, the anti-infective is an antiviral drug. In some embodiments, the anti-infective is an anti-retroviral (ARV). In some embodiments, the ARV is selected from a viral integrase inhibitor, entry inhibitor, non-nucleoside reverse transcriptase inhibitor, or a combination thereof. In some embodiments the viral integrase inhibitor is selected from raltegravir (RAL), the entry inhibitor is selected from maraviroc (MVC), the non-nucleoside reverse transcriptase inhibitor is selected from etravirine (ETR) and dapivirine (DPV), or a combination thereof.

In some embodiments, the anti-infective is an antiviral drug that inhibits herpes simplex virus (HSV) selected from helicase primase inhibitors including pritelivir and a nucleoside analog including acyclovir.

In some embodiments, the pharmaceutical composition inside the polymer core ranges from about 0.5 mg to about 200 mg. In some embodiments, the pharmaceutical composition is released from the drug delivery system at an effective level for up to about ten years. In some embodiments, the effective level ranges from about 1 μg to about 500 μg a day.

In some embodiments, the IUS having one or more drug delivery systems is integrated with an intrauterine frame.

In some embodiments, the drug delivery system is a first drug delivery system, and the IUS further includes a plurality of drug delivery systems. In some embodiments, each drug delivery system includes a polymer core, where the pharmaceutical composition is dispersed within the polymer core. In some embodiments, each drug delivery system further comprises a sheath membrane surrounding the polymer core. In some embodiments, each drug delivery system releases a distinct pharmaceutical composition or active compound.

In some embodiments, the intrauterine frame is an intrauterine device selected from a hormonal IUD or a nonhormonal IUD.

In another aspect, disclosed herein is an intrauterine device (IUD) configured to prevent pregnancy, including pharmaceutical composition, and a delivery system configured to deliver the pharmaceutical composition to a reproductive tract (cervix, uterus/endometrium, fallopian tubes), a lower female genital tract (vaginal, vulvar, vestibule and vestibular glands, and vulva), an anus/rectum, or a combination thereof.

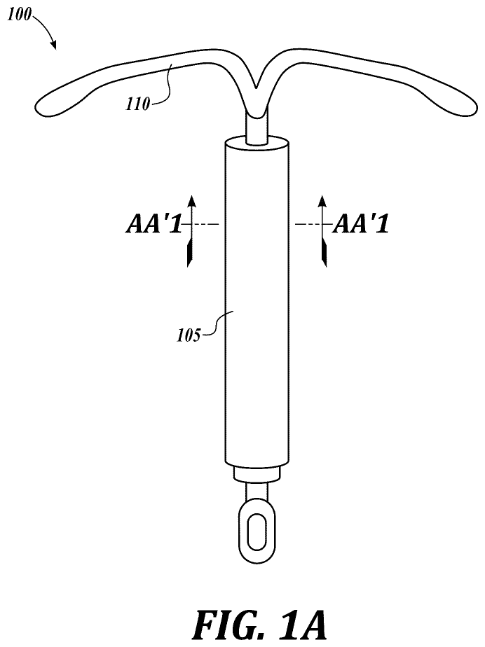

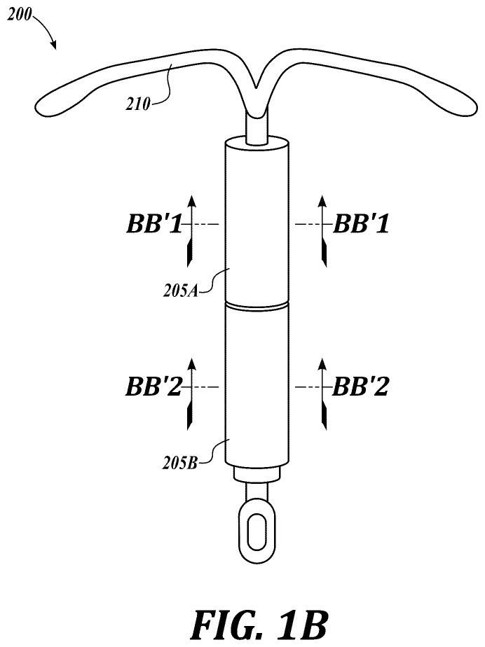

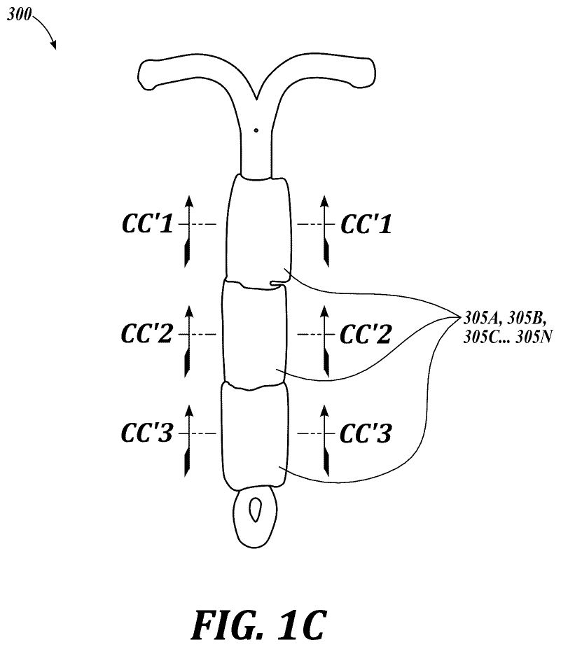

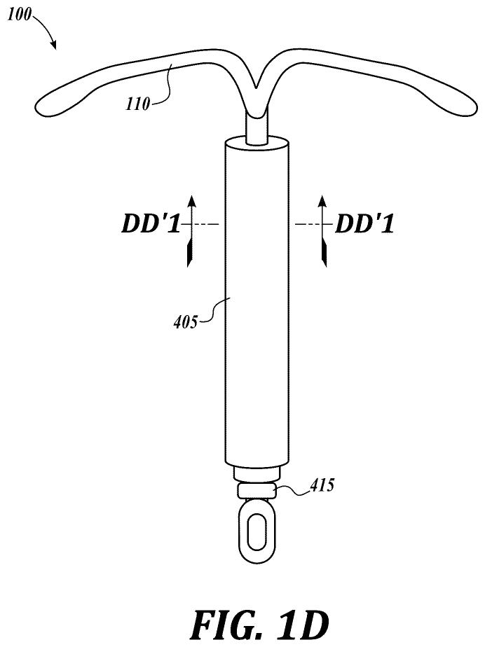

FIG. 1A is an example intrauterine system (IUS) 100 with an example drug delivery system 105, in accordance with the present technology. In some embodiments, the IUS 100 further includes a frame 110 (or intrauterine frame 110). As used herein, the intrauterine frame includes, but is not limited to, a bioinert polyethylene polymer frame, a wire frame, or a combination thereof. While the IUS 100 of FIG. 1A includes a frame 110, it should be understood that in some embodiments, the IUS 100 does not include a frame 110.

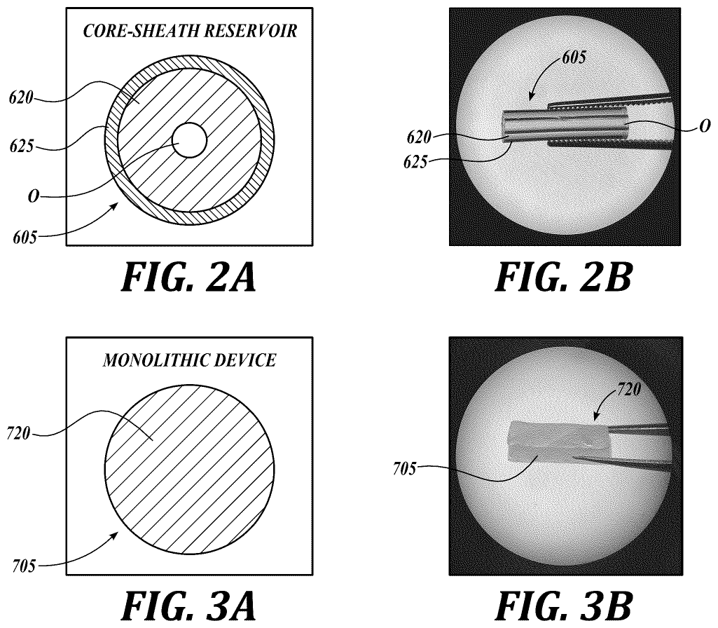

The drug delivery system may take a number of forms, as shown and described in detail in FIGS. 2A-2B and 3A-3B herein. A cross-section line AA′ is illustrated across the drug delivery system 105. Example cross-sections of the drug delivery system 105 are shown and described in detail in FIGS. 2A-2B and 3A-3B.

In some embodiments, as shown and described in detail herein, the drug delivery system 105 is configured to deliver one or more active compounds (or one or more pharmaceutical compositions) to non-systemic extrauterine biological targets. In some embodiments, the biological targets may be in the lower female genital tract (vaginal, vulvar, vestibule and vestibular glands, and vulva; LFGT) and/or anus/rectum of a patient. In some embodiments, the intrauterine system 100 is configured to facilitate uterine-to-LFGT transport of the one or more active compounds, and wherein the one or more active compounds are detectable in tissue, local secretions, or a combination thereof at least 7 days from retention of the intrauterine system into a uterus. In some embodiments, the tissue is selected from endometrial/uterine tissue, cervical tissue, LFGT (vaginal, vulvar, vestibule and vestibular glands, and vulva) tissue, anal/rectal tissue, or a combination thereof.

In some embodiments, the delivery system is further configured to accumulate the one or more active compounds in the LFGT (vaginal, vulvar, vestibule and vestibular glands, and vulva) and/or the anus/rectum for an extended duration. As used herein, the term “extended duration” includes, but is not limited to, at least two weeks, one month, at least two months, at least six months, at least one year, at least two years, or at least 5 years. In some embodiments, the one or more active compounds have a steady state drug concentration of at least 50 ng/mg in the tissue of a patient. In some embodiments, the one or more active compounds have a steady-state drug concentration of at least 0.5-25 ng/mL in the local secretions. In some embodiments, the one or more active compounds have a steady-state drug concentration of at least 100 ng/mL in the local secretions. In some embodiments, the one or more active compounds have a steady-state drug concentration of 0.5-500 ng/mL in the local secretions.

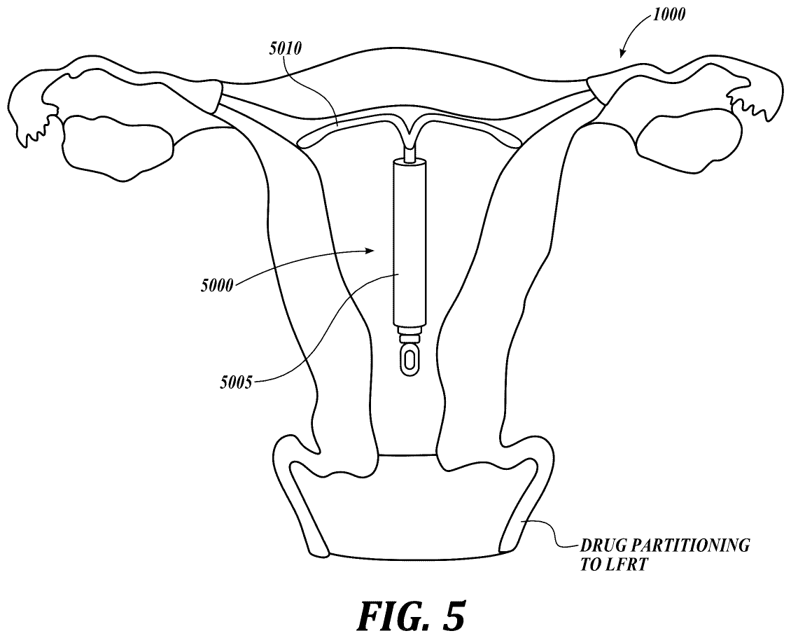

In some embodiments, the drug delivery system 105 is configured to allow uterine-to-vaginal drug (or one or more active compounds) transport to the LFGT (vaginal, vulvar, vestibule and vestibular glands, and vulva) and/or anus/rectum. The human uterus is connected to the vagina by the cervical canal, an elliptical cavity that is fused circumferentially to the vaginal exocervix and uterine endocervix. When properly placed, the IUS 100 sits directly superior to the endocervix at the uterine isthmus (an example of which is shown and described in FIG. 5); thus, any pharmaceutical agent (i.e., one or more active compounds) released from the drug delivery system 105 is directly exposed to tissue and fluid adjacent to the cervical canal. In some embodiments, the IUS 100 includes a hormonal IUD frame (such as frame 310 having one or more contraceptive elements, such as contraceptive element 415). In addition to transport through the cervical canal, direct absorption of IUS 100 delivered drugs by uterine tissue and redistribution through systemic circulation may contribute to uterine-to-vaginal drug transport. Other mechanisms of transport, such as direct diffusion through tissue and counter current exchange between the uterine venous plexus and vaginal artery, are also substantiated by basic anatomical parameters.

In some embodiments, the one or more active compounds are configured to treat one or more sexually transmitted infections (STIs). In some embodiments, the one or more STIs are selected from human immunodeficiency virus (HIV), herpes, chlamydia, gonorrhea, syphilis, and bacterial vaginosis. In some embodiments, the one or more active compounds are a pharmaceutical composition. Representative pharmaceutical compositions include, but are not limited to, anti-infective pharmaceutical compositions, antiviral pharmaceutical compositions, and anti-retroviral (ARV) pharmaceutical compositions. In some embodiments, the ARV pharmaceutical compositions include, but are not limited to raltegravir (RAL), maraviroc (MVC), etravirine (ETR), dapivirine (DPV), or a combination thereof. In some embodiments, the anti-infective pharmaceutical composition is an antiviral pharmaceutical composition configured to inhibit HSV. The antiviral pharmaceutical composition may be one or more nucleoside analogs. In some embodiments, the antiviral pharmaceutical composition may be pritelivir, acyclovir, or a combination thereof. In some embodiments, the pharmaceutical compound is doxycycline. In some embodiments, the pharmaceutical compound is an anti-cancer drug. In some embodiments, the pharmaceutical composition is released from the delivery system at an effective level for up to about ten years. In some embodiments, the effective level ranges from about 1 μg to about 500 μg a day.

In some embodiments, the drug delivery system 105 includes a polymer core, where the one or more active compounds are dispersed within the polymer core. In some embodiments, the drug delivery system 105 further includes a sheath membrane surrounding the polymer core. It should be understood that when a drug delivery system has a polymer core, but not a sheath, it is a “monolithic” or “matrix” drug delivery system. Further, when a drug delivery system has a polymer core and a sheath, it is a “core-sheath reservoir” or a “reservoir” drug delivery system. The matrix drug delivery systems and core-sheath reservoir drug delivery systems are shown and described in detail in FIGS. 2A-2B and 3A-3B.

FIG. 1B is another example intrauterine system 200 with two drug delivery systems 205A, 205B, in accordance with the present technology. In some embodiments, the intrauterine system (IUS) 200 includes a first drug delivery system 205A, a second drug delivery system 205B, and a frame 210.

In some embodiments, the frame 210 is configured to retain the first drug delivery system 205A and/or the second drug delivery system 205B into a uterus. In some embodiments, the IUS may not include the frame 210.

In some embodiments, the delivery system may be a first drug delivery system 205A and a second drug delivery system 205B. The first drug delivery system 205A and the second drug delivery system 205B may be formed of the same materials, or different materials, and shown and described in FIGS. 2A-2B and 3A-3B. Also shown is a cross-section line BB′1 bisecting the first drug delivery system 205A and another cross-section line BB′2 bisecting the second drug delivery system 205B. Example cross-sections of both the first drug delivery system 205A and the second drug delivery system 205B are shown and described in detail in FIGS. 2A-2B and 3A-3B.

In some embodiments, the first drug delivery system 205A is configured to deliver one active compound, and the second drug delivery system 205B is configured to deliver a second active compound. In some embodiments, the first drug delivery system 205A is configured to deliver a first pharmaceutical composition, as described herein, and the second drug delivery system 205B is configured to deliver a second pharmaceutical composition. The first pharmaceutical composition and the second pharmaceutical composition may be any of the pharmaceutical compositions described herein. In some embodiments, each drug delivery system 205A, 205B is configured to deliver the same pharmaceutical composition.

In some embodiments, the first drug delivery system 205A and/or the second drug delivery system 205B includes a polymer core, where the one or more active compounds is dispersed within the polymer core. In some embodiments, the first drug delivery system 205A and/or the second drug delivery system 205B further includes a sheath membrane surrounding the polymer core. It should be understood that when a delivery system has a polymer core, but not a sheath, it is a “monolithic” or “matrix” delivery system. Further, when a delivery system has a polymer core and a sheath, it is a “core-sheath reservoir” or a “reservoir” delivery system. The matrix delivery systems and core-sheath reservoir delivery systems are shown and described in detail in FIGS. 2A-2B and 3A-3B.

FIG. 1C is yet another example intrauterine system 300 having a plurality of drug delivery systems 305A, 305B, 305C . . . 305N, in accordance with the present technology. In some embodiments, the intrauterine system (IUS) 300 further includes a frame 410.

In some embodiments, the IUS 300 can have any number of drug delivery systems. While three drug delivery systems are illustrated in the plurality of drug delivery systems 305A, 305B, 305C . . . 305N, one skilled in the art will recognize any number of drug delivery systems may be incorporated into the IUS 300.

In some embodiments, each delivery system of the plurality of drug delivery systems 305A, 305B, 305C . . . 305N may be formed of the same materials, or different materials, and shown and described in FIGS. 2A-2B and 3A-3B. Also shown are cross-section lines CC′1, CC′2, CC′3 of each drug delivery system of the plurality of drug delivery systems 305A, 305B, 305C . . . 305N. Example cross-sections of the plurality of drug delivery systems 305A, 305B, 305C . . . 305N are shown and described in detail in FIGS. 2A-2B and 3A-3B.

In some embodiments, each drug delivery system of the plurality of drug delivery systems 305A, 305B, 305C . . . 305N includes a polymer core, where the one or more active compounds is dispersed within the polymer core. In some embodiments, each drug delivery system of the plurality of drug delivery systems 305A, 305B, 305C . . . 305N further includes a sheath membrane surrounding the polymer core. It should be understood that when a drug delivery system has a polymer core, but not a sheath, it is a “monolithic” or “matrix” drug delivery system. Further, when a drug delivery system has a polymer core and a sheath, it is a “core-sheath reservoir” or a “reservoir” drug delivery system. The matrix drug delivery systems and core-sheath reservoir drug delivery systems are shown and described in detail in FIGS. 2A-2B and 3A-3B.

In some embodiments, each drug delivery system of the plurality of drug delivery systems 305A, 305B, 305C . . . 305N is configured to deliver one or more active compounds. In some embodiments, each drug delivery system of the plurality of drug delivery systems 305A, 305B, 305C . . . 305N is configured to deliver a pharmaceutical composition. In some embodiments, each drug delivery system of the plurality of drug delivery systems 305A, 305B, 305C . . . 305N is configured to deliver one or more active compounds distinct from one another, i.e., a first drug delivery system delivers a first compound, a second drug delivery system delivers a second compound, and so on. In some embodiments, each drug delivery system of the plurality of drug delivery systems 305A, 305B, 305C . . . 305N releases a different active compound of the one or more active compounds. In some embodiments, each drug delivery system of the plurality of drug delivery systems 305A, 305B, 305C . . . 305N is configured to deliver the same one or more active compounds. In some embodiments, each drug delivery system of the plurality of drug delivery systems 305A, 305B, 305C . . . 305N are configured to deliver any of the pharmaceutical compositions described herein.

FIG. 1D is yet another example intrauterine system 400 having one or more contraceptive elements 415, in accordance with the present technology. In some embodiments, the contraceptive elements 415 is one or more hormonal or nonhormonal elements.

In some embodiments, the IUS 400 is further configured to prevent pregnancy. In some embodiments, the one or more contraceptive elements 415 are configured to prevent pregnancy. In some embodiments, the one or more contraceptive elements 415 are copper ferrules. In some embodiments, the one or more contraceptive elements 415 are spermicidal. One skilled in the art should recognize that in addition to or instead of the one or more contraceptive elements 415, additional contraceptive mechanisms may be used, such as, for example, hormones. The one or more contraceptive elements 415 may be coupled to the frame 410 at a top of the frame 410 and/or below the delivery system 405.

In some embodiments, the drug delivery system 405 includes a polymer core, where the one or more active compounds are dispersed within the polymer core. In some embodiments, the drug delivery system 405 further includes a sheath membrane surrounding the polymer core. It should be understood that when a drug delivery system has a polymer core, but not a sheath, it is a “monolithic” or “matrix” drug delivery system. Further, when a drug delivery system has a polymer core and a sheath, it is a “core-sheath reservoir” or a “reservoir” drug delivery system. The matrix drug delivery systems and core-sheath reservoir drug delivery systems are shown and described in detail in FIGS. 2A-2B and 3A-3B.

While FIGS. 1A-1D show multiple example intrauterine systems 100, 200, 300, 400 having various components, one skilled in the art will recognize that the specific components illustrated in each of the FIGS. 1A-1E are not limiting. That is, any of the intrauterine systems 100, 200, 300, 400 illustrated may include any of the components shown in any other embodiment.

Two drug delivery systems were developed to deliver the one or more active compounds: the monolithic matrix and the core-sheath reservoir. Each has the potential to deliver physiochemically distinct ARVs to the LFGT.

FIGS. 2A-2B is an example cross-section and perspective view of drug delivery systems 605, respectively, in accordance with the present technology. It should be understood that example drug delivery system 605 may be any of the drug delivery systems illustrated herein, including drug delivery system 105, drug delivery system 205, first drug delivery system 305A, second drug delivery system 305B, any drug delivery system of the plurality of drug delivery systems 305A, 305B, 305C . . . 305N, and drug delivery system 405. In some embodiments, drug delivery system 605 may be independent of a frame or coupled to a frame (such as frames 110, 210, 310, 410) as shown in FIGS. 1A-1D.

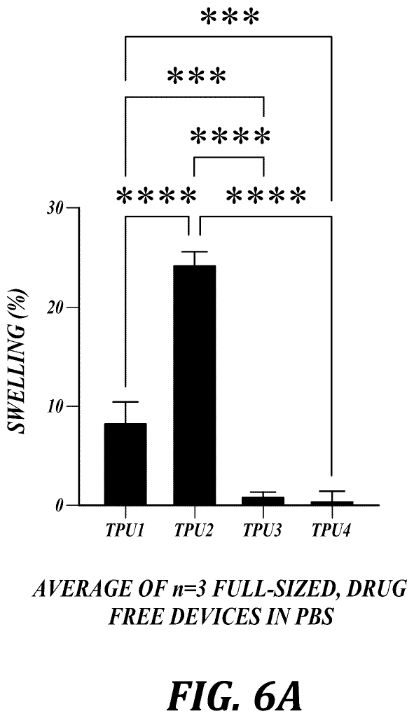

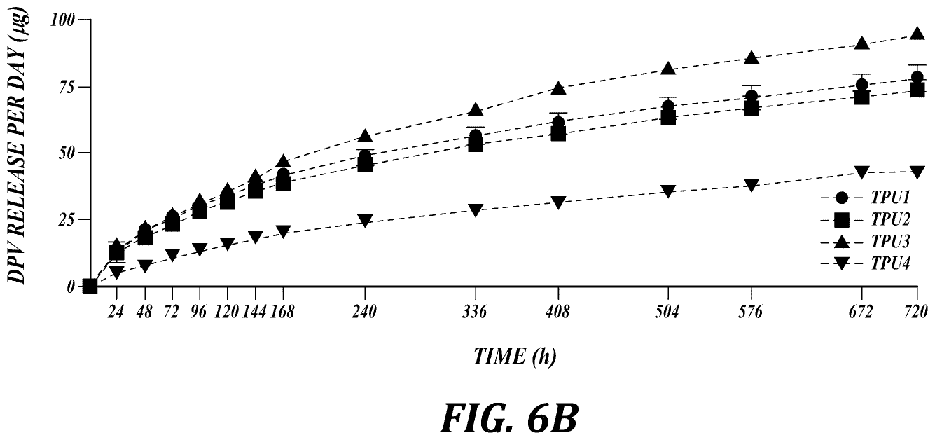

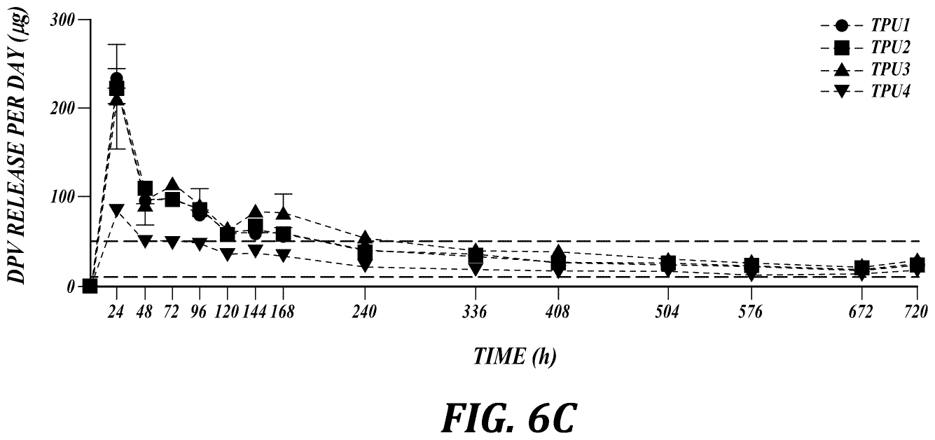

The drug delivery system 605 shown in FIGS. 6A-6B may be referred to as a “sheath-core reservoir” drug delivery system or a “reservoir” drug delivery system. In some embodiments, the drug delivery system 605 shown in FIG. 2A is a cross section along any one of the cross-section lines illustrated herein (AA′, BB′1, BB′2, CC′2, CC′2, CC′3, DD′1).

In some embodiments, the drug delivery system 605 includes a core 620 formed of a core polymer. In some embodiments, the one or more active compounds are dispersed with the core 620. In some embodiments, the core polymer is selected from thermoplastic polyurethanes (TPUs), which are synthetic linear segmented block copolymers with high material flexibility and biocompatibility. TPUs have varying degrees of water uptake and crystallinity that synergistically modulate drug release kinetics. Using iterative in vitro drug release testing coupled with mechanistic model-informed in silico predictions, we a formulation workflow was developed to rapidly design delivery systems to meet durable sustained release target specifications. In some embodiments, the core polymer is hydrophobic. In some embodiments, the core polymer is a polyether. In some embodiments, the core polymer is PY-PT43DE20. In some embodiments, the core 620 has a crystalline structure. In some embodiments, the core 620 is configured to have about 2-20% water uptake.

In some embodiments, the drug delivery system 605 further includes a sheath membrane 625 surrounding the core 620. In some embodiments, the sheath membrane 625 has a thickness T between about 50 μm to about 500 μm. In some embodiments, the sheath membrane 625 has a thickness T of 100 μm. In some embodiments, the sheath membrane 625 has a thickness of 200 μm. In some embodiments, the sheath membrane 625 is formed of a sheath polymer. In some embodiments, the sheath polymer is different from the core polymer. In some embodiments, the sheath polymer is hydrophobic. In some embodiments, the sheath polymer is TPU. In some embodiments, the sheath polymer is a polyether. In some embodiments, the sheath polymer is PY-PT72AE. In some embodiments, the sheath polymer is a polycarbonate. In some embodiments, the sheath polymer is PY-PN73AE. In some embodiments, the sheath 625 has a semi-crystalline form. In some embodiments, the sheath 625 is configured to have less than 2% water uptake.

In some embodiments, the drug delivery system 605 may further include an opening O. In some embodiments, a frame (such as frame 110, 210, 310, 410) of an IUS (such as IUS 100, 200, 300, 400) is configured to thread through the opening O.

FIGS. 3A-3B is an example cross-section and perspective view of drug delivery systems 705, respectively, in accordance with the present technology. It should be understood that example drug delivery system 705 may be any of the drug delivery systems illustrated herein, including drug delivery system 105, drug delivery system 205, first drug delivery system 305A, second drug delivery system 305B, any drug delivery system of the plurality of drug delivery systems 305A, 305B, 305C . . . 305N, and any drug delivery system of the plurality of drug delivery systems 505A, 505B, 505C . . . 505N. In some embodiments, drug delivery system 705 may be independent of a frame (such as frames 110, 210, 310, 410, 510) or coupled to a frame as shown in FIGS. 1A-1E to form an IUS.

Shown in FIGS. 3A-3B is a “monolithic” or “matrix” drug delivery system. In some embodiments, the drug delivery system 705 shown in FIG. 2A is a cross section along any one of the cross-section lines illustrated herein (AA′, BB′1, BB′2, CC′1, CC′2, CC′1, CC′2, CC′3, DD′1). In some embodiments, the drug delivery system 705 includes a core 620 formed of a core polymer. In some embodiments, the one or more active compounds are dispersed with the core 620. In some embodiments, the core polymer is thermoplastic polyurethane (TPU). In some embodiments, the core polymer is hydrophobic. In some embodiments, the core polymer is PY-PT43DE20. In some embodiments, the core polymer is a polyether. In some embodiments, the core 620 has a crystalline structure.

The drug delivery system 705 does not include a sheath membrane (such as sheath membrane 625). When the delivery system does not include a sheath membrane, it is referred to as a “monolithic” delivery system or a “matrix” drug delivery system 705. The drug delivery system 705 may diffuse one or more active compounds through a polymer network (such as with a Fickian diffusion).

While not illustrated in FIGS. 3A-3B, in some embodiments, the matrix drug delivery system 705 includes an opening (such as opening O), but in other embodiments, the opening may be omitted. In some embodiments, the matrix delivery system may be coupled to a frame (such as frame 110, 210, 310, 410) of an IUS (such as IUS 100, 200, 300, 400) by penetrating the drug delivery system 705 with the frame. In other embodiments, the drug delivery system 705 may be molded around the frame.

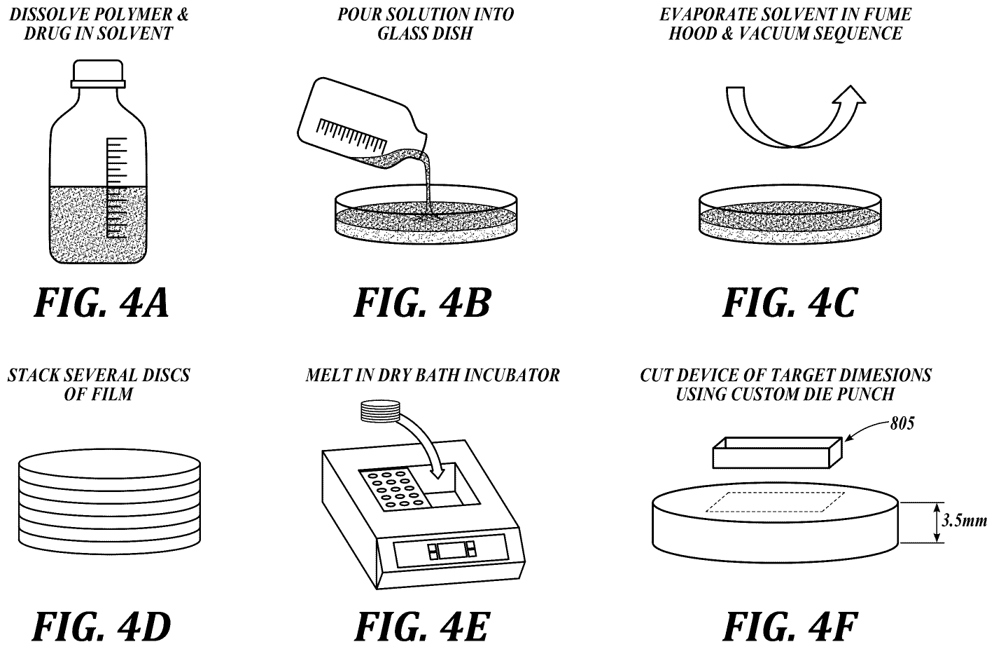

FIGS. 4A-4F are example process steps of preparing a drug delivery system 705, in accordance with the present technology.

In FIG. 4A polymer (i.e., a core polymer, such as core polymers 620, 720) and one or more active compounds (a pharmaceutical compound or “drug”) are dissolved in a solvent. In some embodiments, the polymers are selected from TPUs, such as PY-PT42DE35 (referred to herein as “TPU1”), PY-PT83AE35 (referred to herein as “TPU2”), PY-PT72AE (referred to herein as “TPU3”), and PYPT87AE (referred to herein as “TPU4”).

In FIG. 4B, the resulting solution of the dissolved polymer and one or more active compounds is poured into a container. In some embodiments, the container is a glass dish.

In FIG. 4C, the solvent is evaporated. In some embodiments, the solvent is evaporated in a fume hood. In some embodiments, a vacuum sequence may be used to evaporate the solvent. In some embodiments, by evaporating the solvent, the solution becomes a film.

In FIG. 4D, several films are stacked. In some embodiments, each film may take the form of the container the solution was poured into. In some embodiments, the films may be discs of film.

In FIG. 4E, the films are melted. In some embodiments, the films are melted with a dry bath incubator.

Finally, in FIG. 4D, a drug delivery system 805 (or “device”) is cut from the melted films. In some embodiments, the drug delivery system 805 is cut using a custom die punch. In some embodiments, the drug delivery system 805 may have a thickness of 3.5 mm. As explained herein, the drug delivery system 805 may have a thickness ranging from 10 μm to 500 μm.

FIG. 5 is a diagram of an example intrauterine system 5000 retained in a uterus 1000, in accordance with the present technology. In some embodiments, the intrauterine system is configured to be retained in a uterus 1000 of a patient. In some embodiments, the intrauterine system described herein is an intrauterine device (IUD) 5000, having a delivery system 5005, a frame 5010, and a one or more contraceptive elements (such as contraceptive element 415).

By retaining the IUS 5000 in the uterus 1000, the IUS 5000 may simultaneously prevent pregnancy, and deliver one or more active compounds to biological targets in the LFGT and/or rectum of the patient.

In another aspect, disclosed herein is a method of using the intrauterine system, or in one example, the IUS 5000, described herein. In some embodiments the method includes retaining the intrauterine system in a uterus and delivering the one or more active compounds to non-systemic extrauterine biological targets in the LFGT or the anus/rectum.

In some embodiments, delivering the one or more active compounds includes accumulating the one or more active compounds in the LFGT or anus/rectum for at least one year. In some embodiments, delivering the one or more active compounds includes accumulating a steady-state drug concentration of the one or more active compounds of at least 50 ng/mg in tissue, a steady-state drug concentration of the one or more active compounds of at least 100 ng/ml in local secretions, or a combination thereof. In some embodiments, delivering the one or more active compounds includes accumulating a steady-state drug concentration of the one or more active compounds of at least 0.5 to 25 ng/mg in tissue. In some embodiments, delivering the one or more active compounds includes accumulating a steady-state drug concentration of the one or more active compounds of 0.5-200 ng/mg in tissue. In some embodiments, delivering the one or more active compounds includes accumulating a steady-state drug concentration of the one or more active compounds of at least 0.5-25 ng/ml in local secretions. In some embodiments, delivering the one or more active compounds includes accumulating a steady-state drug concentration of the one or more active compounds of 0.5 to 500 ng/mL in local secretions.

In some embodiments, the method further includes treating one or more sexually transmitted infections with the one or more active compounds and/or the pharmaceutical compounds described herein.

### EXAMPLES

The development of long-acting, discreet, adherence-independent and female controlled multipurpose prevention technology is critical in the effort to prevent sexually transmitted infections including HIV and unwanted pregnancy among people with female reproductive anatomy. Long-acting reversible contraception, particularly IUDs, demonstrate delivery of active agents locally to the female reproductive tract at sustained doses for up to eight years in duration, and emerging IUD frame designs are amenable to integration of an additional, orthogonal drug delivery system to supplement their contraceptive mechanism. Here, the design and integration of two candidate drug delivery systems is illustrated, the monolithic matrix delivery system (as illustrated in FIGS. 3A-3B) and core-sheath reservoir delivery system (as illustrated in FIGS. 2A-2B), onto a frame copper IUD. Various thermoplastic polyurethanes were investigated for sustained release of physiochemically unique ARVs and developed an in-vitro-in-silico workflow to optimize formulation parameters for targeted release kinetics. While IUDs offer in-utero release of encapsulated agents, the feasibility of local drug transport from the upper to lower FRT, where ARV is needed to prevent HIV infection, was not known. Safety and pharmacokinetic study of ARV-IUDs was investigated in non-human primates and it was found that intrauterine device placement results in high, clinically relevant concentrations of ARV in the upper (cervix, uterus/endometrium, fallopian tubes) and lower (vaginal, vulvar, vestibule and vestibular glands, and vulva) organs but low plasma drug concentrations. Residual drug analysis of matrix and reservoir delivery systems suggested sustained release of ARV for up to one year, potentiating this device as an effective long-acting, adherence-free, discreet and female-controlled MPT.

Aliphatic polyether TPUs were initially selected for this drug delivery system due to their resistance to hydrolysis and microbial growth compared to polyester-based TPUs. Specifically investigated herein is PY-PT42DE35 (referred to herein as “TPU1”), PY-PT83AE35 (referred to herein as “TPU2”), PY-PT72AE (referred to herein as “TPU3”), and PYPT87AE (referred to herein as “TPU4”).

Hardness, water uptake and hydrophobicity can modulate the release rate of encapsulated active pharmaceutical ingredient (API), as demonstrated by the in vitro release data in which low loading (1 wt %) ARV were screened in various TPUs. Here, TPU3 was the least crystalline TPU assayed as determined by a shore durometer which reflects the ratio of hard to soft segments in a copolymer. Long, low polarity segments are called “soft” while short, high polarity segments are characterized as “hard.” The high polarity of hard segments results in greater aggregation and crystallization, which can reduce the permeability, water uptake, and/or the diffusion coefficient of ARV within the polymer matrix. It was found that TPU3 offered the most rapid release of DPV, more so than either swellable TPU; as such it was inferred that the diffusion coefficient of DPV through a wetter TPU3 matrix is significantly greater than that of other TPUs. This finding is in accordance with various studies that have shown that an increase in the soft to hard segment ratio results in an increase in the release rate of encapsulated drug. Water uptake and hydrophobicity are closely related and generally result in an increase in swelling of the polymer matrix, which accelerates drug release by increasing the porosity and free volume for diffusion. This was observed in the similarly rapid drug release of DPV from TPU1 and TPU2, the two hydrophilic matrices investigated here, and they rapid dissolution of raltegravir (RAL), maraviroc (MVC), and etravirine (ETR) from TPU2 in comparison to the nonswelling TPU3 and TPU4. In sum, in vitro release data shows that the relative dissolution profiles of each TPU agreed with our understanding of release kinetics from matrices with variable crystallinity and water uptake characteristics. The Korsmeyer-Peppas model allowed us to further validate the observed release kinetics of the matrix delivery system prototypes. Anomalous release is an appropriate description of the drug release mechanism of these TPUs based on their variable, nonzero water uptake and non-erosion properties. Accordingly, in these TPU matrices, the diffusion rate and polymer relaxation rate are of the same order of magnitude.

The most crystalline and hydrophobic TPU, TPU4, consistently released all low-loading ARVs the slowest. Despite its relative performance at offering sustained release compared to other TPUs, TPU4 still exhibited discreet differences in dissolution kinetics among each ARV. For instance, RAL, the most hydrophilic ARV, released the slowest from the hydrophobic TPU4 matrix despite its more rapid dissolution from TPU 2 and 3 compared to MVC and ETR. Korsmeyer-Peppas modelling for RAL in TPU3 and 4 indicates Quasi-Fickian drug transport wherein drug release is mediated by non-swellable matrix diffusion. Thus, the diffusion coefficient of RAL in each polymer is a significant mediator of dissolution. It can then be inferred that RAL, being highly hydrophilic, is less compatible with hydrophobic TPU4 than the other hydrophobic ARVs and therefore exbibits slower diffusion through the matrix. This discussion highlights a unique challenge in MPT development, where multiple distinct pharmaceutical agents are incorporated, as physicochemical characteristics of a drug are known to play a significant role in drug release. The drug-polymer interaction, described by the diffusion coefficient and solubility of an ARV within a PU matrix, as well as drug loading and partitioning can modulate the rate and kinetics of drug dissolution. A notable benefit of the ARV-IUD design is the ability to incorporate multiple distinct delivery systems onto a single IUD frame wherein each delivery system can be optimized individually to suit the release specifications of a single ARV. Modular construction of an IUD with uniquely formulated units releasing pharmaceutical agents orthogonally opens the door to more rapid development of MPT for a variety of prophylactic or therapeutic targets.

Comparing the relative cumulative release of multiple ARVs loaded at a low weight percent in various TPUs proved an effective method of selecting an optimal TPU for evaluating matrices loaded with a greater amount of drug. However, further quantification using mechanistic models of drug release from unsaturated and saturated matrices enhanced this optimization process significantly and identified precise drug loading requirements capable of achieving target release durations for the triple-ARV combination. This analysis predicted loading requirements for ARVs in the leading TPU candidate, TPU4, and upon validation we confirmed that RAL loaded at 30 wt % in a TPU4 matrix delivery system can offer durable release for approximately one year and MVC loaded at 30 wt % in TPU4 can achieve approximately two years of release, similar to ETR loaded at 16.6 wt %. As such, it is possible to fabricate monolithic dispersions of three physicochemically distinct ARVs in the same TPU that can release each drug for at least one year and up to nearly 2.5 years. This workflow also confirmed that a TPU matrix delivery system with this geometry is not capable of delivering DPV for a sustained period. This strongly motivated the development of a reservoir-based delivery system for DPV. Ultimately, this modelling workflow provides an efficient and relatively high-fidelity method of evaluating monolithic matrix delivery systems, relying on only two data sets for a given drug-polymer combination—below and above saturation solubility—to extrapolate precise loading conditions to achieve any sustained-release specification.

A method of evaluating core-sheath reservoir formulations that varied based on their sheath polymer chemistry and thickness was further demonstrated. The significance of these two properties on drug release from a core-sheath reservoir delivery system is mathematically described by Baker and Lonsdale's derivation of the radial flux of drug from a cylindrical reservoir. Sheath polymer chemistry affects drug release based on its relationship to drug diffusion and drug partitioning kinetics at the core-sheath interface; a sheath that is more permeable to API and offers more favorable partitioning for a given core polymer will release drug faster. Conversely, given a constant outer radius, a thicker sheath membrane results in in slower drug release due to the inverse relationship of flux with the natural log of the ratio of outer to inner radius. In assessing in vitro release of DPV loaded at 1% in the core of various reservoir delivery systems, it was observed that polyether sheaths resulted in more rapid DPV release than polycarbonate sheaths and the data confirmed the expectation that thicker sheaths resulted in a slower rate of drug release. Unexpectedly, when using release data from unsaturated (low-loading) reservoirs to predict release kinetics from saturated (high-loading reservoirs), it was observed that R3 (100 μm, polycarbonate sheath) was expected to surpass R2 (200 μm, polyether sheath) in its cumulative release from a saturated reservoir despite its slightly slower release from the unsaturated reservoir. Accordingly, in reservoirs with nonconstant activity sources (unsaturated), sheath polymer chemistry plays a more significant role in mediating drug release than in reservoirs with constant activity sources. In the latter condition, sheath polymer thickness played a dominant role as the two reservoir delivery system prototypes with 100 μm thick sheaths (R1 & R3) were predicted to release drug the fastest. All reservoirs except R1 could be loaded below a theoretical 50 wt % loading threshold. However, the relevance of this maximum loading threshold is perhaps less significant for a reservoir delivery system than a matrix delivery system, as total device loading is lower than the core polymer loading and the sheath polymer can provide mechanical stability that a highly loaded core may lack. Indeed, based on mass of drug loaded, 58 mg of DPV loaded in R1 is similar to the 52 mg of LNG loaded into a Mirena IUD. All of the highly loaded reservoir delivery system prototypes would release drug above the 10 μg/day minimum, with R1, R2, and R3 exceeding the 50 μg/day target for one-year of sustained release. In sum, the reservoir delivery system modelling workflow demonstrated that all reservoir formulations were theoretically capable of releasing drug for one year with characteristics that match or are relatively similar to the initial design specifications.

Ultimately, two polycarbonate reservoirs were selected to load with a model-informed 30 wt % DPV and assessed in vitro release. Reservoirs with saturated cores exhibited more rapid release in the first week of dissolution than the remaining three weeks. This is likely due to burst effects caused by equilibration of DPV across the core and membrane polymer that results in the reservoir initially behaving as a matrix device. Indeed, devices were incubated post-fabrication for 2 weeks at 40° C. in order to achieve DPV equilibration as described and prevent the opposite early-time phenomena called “lag-time” effects. Lag-time effects occur right after dosage form preparation when the sheath membrane is devoid of drug, requiring time for drug diffusion and resulting in a slower initial drug release rate. Ultimately, both polycarbonate sheath reservoirs were estimated to achieve greater than one-year of release while exhibiting zero-order release kinetics indicative of consistent daily drug release. As such, both formulations are promising delivery system candidates for the ARV-IUD system. This data validated our approach of screening low loading reservoir formulations to inform delivery system formulation parameters using a mechanistic model for drug release from reservoir devices.

In vitro dissolution testing is useful for understanding drug release mechanisms and comparatively evaluating delivery system formulations; however, it is limited in its ability to recapitulate in vivo release kinetics. Conditions of in vitro release simulate the in vivo environment by controlling parameters such as temperature, pH, sink conditions, and aqueous conditions. Simulated conditions do not account for the low-flow environment in vivo, dynamics of protein binding, or metabolic effects. A non-ionic surfactant is used to increase the solubility of DPV, MVC, and ETR in release media to ensure sink conditions. Drug solubility in dissolution media is directly proportional to particle dissolution; as such, in vitro dissolution testing conditions here may have accelerated drug release. However, the two-fold acceleration in release, which equates to approximately 20 μg/day, matches the 20 μg/day in vivo release rate of the Mirena IUD, as determined by clinical studies. In a clinical pharmacology review by the Center for Drug Evaluation and Research, an in vitro-in vivo correlation (IVIVC) was described for the Mirena IUD in which the dissolution profiles of LNG tested in vitro and in vivo (ex vivo) correlated with a slope of 1.165, indicating a nearly directly proportional relationship. The in vitro dissolution conditions were not described. Based on these reports, it appears possible that “accelerated” in vitro dissolution due to increased solubility does not necessarily preclude interpretation of data with regard to in vivo performance. This in vitro release data and consideration for previously reported in utero release kinetics allows us to conclude that we developed matrix and reservoir delivery systems that are promising for delivery of physicochemically distinct ARVs for several months to years and that our in vitro delivery system optimization schemes are extensible to formulation of additional ARVs and APIs. However, the dissolution of leading matrix and reservoir delivery system candidates were evaluated in utero for a more exact understanding of the duration and kinetics of drug release.

A baboon non-human primate model was selected for pharmacokinetic evaluation of the delivery system because baboons have reproductive anatomy that is similar to humans with short rectilinear cervical canals that allow for nonsurgical, transcervical IUD implantation. Compared to macaques, baboons have a larger uterus that accommodates a human-sized IUD with minimal modification, permitting recapitulation of the biomechanics and positioning of human IUD placement. Upon successful integration of both matrix and reservoir delivery system onto a miniature copper IUD frame, which was amenable to non-surgical insertion in baboon uteri, favorable tolerability and safety was observed. It was noted that there was no weakening or inflammation of the vaginal epithelium after 30-days of ARV-IUD implantation. The non-keratinized, multilayered vaginal mucosa is a feature of innate mucosal immunity; indeed, a weakened vaginal epithelium is a characteristic pathway by which HIV and other STI virions can invade the genital mucosa. Cervical biopsies containing endo- and exocervical epithelium similarly showed minimal atypia or signatures of inflammation. Inflammatory cell infiltrate and some epithelial erosion of the 28-day post-insertion endometrial tissue was noted; however, this observation is in accordance with the expectations regarding insertion and use of a standard IUD. IUD placement involves necessary infliction of trauma on the cervical canal due to dilation and transcervical passage of an insertion tube. Moreover, IUD placement in the uterine cavity invariably results in morphological and cellular changes to the uterine mucosa and endometrium. The IUD is a foreign body; when in contact with the endometrium, the mucosa becomes flattened, atrophic, and eroded and a nonspecific inflammatory response is elicited that consists of focal inflammatory infiltrate and increased local vascularization, as noted in histological analysis. Typically, vaginal tissue morphology is unaffected by the presence of an IUD, whereas cervical tissue often contains inflammatory cells and squamous abnormalities, consistent with the findings. These anti-implantation effects, particularly in the endometrium, are essential to the contraceptive function of the IUD, and are therefore not considered safety concerns of IUD use. The histological responses noted in this study agree with expected changes to tissue morphology upon foreign body implantation.

The IUD frame used in this study is composed of bioinert polyethylene polymer and has a long history of safety and use in non-US markets. The primary safety concern associated with Cu380 Mini is perforation of the uterine corpus or cervix; this most often occurs during insertion; however, the incidence of occurrence is low (1 per 1000 insertions) and similar to that of other IUD types. Therefore, in the context of this study, the IUD frame itself is not expected to contribute to aberrant safety concerns. Similarly, in a safety and pharmacokinetic trial of dapivirine delivery to the vaginal tract by way of an intravaginal ring, no treatment-emergent adverse events were observed in women exposed intravaginal dapivirine for one month, nor does dapivirine exert any toxicity on ex vivo polarized cervical tissue at concentrations up to 1 mM (0.33 mg/mL). Direct administration of antiretroviral agents to the uterine cavity is the only unique intervention implicated in this delivery system that has not yet been investigated insofar as safety. The toxicity of DPV on endometrial tissue explants should be evaluated directly in future study. However, there is cause to assume DPV would be tolerated in the endometrium at similar concentrations to the tolerance of cervicovaginal tissue. Given that histological examination of endometrial, cervical, and vaginal tissue did not deviate from anticipated effects of IUD placement, we conclude that intrauterine delivery of dapivirine is likely safe and well-tolerated in baboons.

The pharmacokinetic evaluation of ARV-IUD installation has significant implications for viability of this dosage form as a PrEP modality and for the understanding of drug transport in the FRT. The vaginal and cervical tissue concentrations measured here are likely the most relevant with respect to the prophylactic potential of this delivery system. In an ex vivo viral inhibition model, dapivirine inhibited productive infection of ectocervical tissue at concentrations as low as 0.1 nM (0.033 ng/mL), while 99% of provirus formation was inhibited at 1 nM (0.33 ng/mL) [41]. At 10 nM (3.3 ng/mL), dapivirine prevented 90% of infection caused by dissemination of HIV-1 by CD4+ dendritic cells, an inhibition mechanism to prevent HIV infection. This inhibitory potency was retained in the presence of biological fluids such as cervicovaginal mucus and whole semen; thus, the IC90 of DPV in cervicovaginal tissue is taken to be 3.3 ng/mL. It is worth noting that this IC90 value reflects the concentration of DPV with which tissue explants were treated, not the measured intracellular concentration of DPV; thus, the intracellular IC90 in tissue may be even less. Here, matrix and reservoir delivery system ARV-IUDs resulted in cervical tissue DPV concentrations that were, on average, nearly 20 and 40-times this IC90, respectively. Similarly, vaginal tissue measured up to 19-times the cervicovaginal IC90, thus suggesting protective potential of this MPT. Given variation in anatomical length scales and mucosal physiology between female baboons and humans, the concentration of drug in cervicovaginal tissue measured here to may not perfectly recapitulate human pharmacokinetics. However, based on the general anatomical and physiological similarities in FRTs, similar partitioning will likely occur in humans and will result in comparable, high levels of DPV in human cervicovaginal tissue upon ARV-IUD instillation.

The significance of high concentrations of DPV measured in endometrial tissue should not be overlooked. While early studies in NHPs have contributed to a commonly held understanding that HIV primarily productively infects the lower FRT, emerging evidence suggests that the upper FRT is also target for HIV infection and the endometrium is particularly susceptible to viral remodeling that promotes productive infection and viral dissemination. The role of the endometrium in HIV infection in vivo remains to be further understood, however, its immunological susceptibility to HIV suggests the potential usefulness of including the upper FRT in tissue targeted for protection against HIV. This study is the first to provide pharmacokinetic evidence of the potential to protect against HIV infection in the endometrium as well as cervicovaginal tissue. The high concentrations of ARV found in endometrial biopsies suggest that this PrEP modality would be highly protective against HIV infection in the upper FRT.

Vaginal secretion concentrations of DPV that were over 1000 times the IC90 in cervicovaginal fluid were measured, which suggests highly protective levels of DPV that agree, in order of magnitude, to those measured in the RING-004 intravaginal DPV ring clinical trial. Not only was the concentration of DPV measured in vaginal secretions significantly higher than the minimum concentration for protection against HIV for matrix and reservoir delivery systems, but it was also higher than the concentrations measured in vaginal, cervical, and endometrial tissue biopsies at day 28 for respective study arms, the latter of which were taken directly adjacent to the delivery system implantation site. This could indicate a greater propensity for transport of small molecules from the upper to lower FRT via the cervical lumen, rather than by direct diffusion through tissue or by direct or countercurrent venous or lymphatic circulation. The dominance of this transport pathway is supported in theory by anatomy and physiology of the female reproductive tract. Indeed, it is known that, by way of myometrial contractions and cervical peristalsis, uterine contents can travel inferiorly through the cervical lumen. This is demonstrated by the processes of menstruation and childbirth. These pharmacokinetic findings could inform delivery system design for other small molecules or biologics for which transport from the uterus to the vaginal tract may have prophylactic or therapeutic potential. Uterine to vaginal drug delivery warrants further investigation, for example by organ explant open perfusion studies such as those which elucidated the reverse vaginal-to-uterine first pass effect, to identify precise biophysical mechanisms responsible for pharmacokinetic outcomes.

The plasma DPV concentrations measured after instillation of matrix and reservoir ARV-IUDs were consistent in order of magnitude with reported values of ARV and hormone in human plasma upon intravaginal ring and hormonal IUD use, respectively. In a clinical trial of DPV vaginal rings in women, it was found that plasma concentrations upon vaginal DPV delivery had a Cₘₐₓ of 355 μg/mL. It was noted that this is well below the concentration required to reduce viral load by oral DPV administration, which is on the order of 100-200 ng/mL, and is a concentration that is unlikely to cause viral DPV-specific resistant mutations. This is not unlike other findings where upon measuring plasma levonorgestrel (LNG) concentrations after administering LNG-IUDs to women, it was found that IUD delivery resulted in plasma LNG concentrations of 202±102 μg/mL, whereas oral LNG dosing with the drug Cyclabil resulted in plasma LNG concentrations of 559±209 μg/mL. While an oral control in this nonhuman primate study was not included, it would be expected that oral DPV would have resulted in comparatively higher plasma drug levels than the ARV-IUD study arms. Given high tissue and vaginal secretion concentrations of DPV, the data suggests that ARV delivered into the uterine space preferentially partitions to local mucosal and tissue compartments while resulting in low systemic exposure. Therefore, DPV delivered in the uterus follows non-systemic routes of transport to distribute in lower FRT tissue, thereby further substantiating the theory of direct uterine-to-vaginal drug transport.

As expected, reservoir delivery systems are poised to offer DPV release on a more sustained timescale compared to matrix delivery systems and the predictions nearly meet the one-year target proposed in the design of this MPT. Moreover, these devices achieved sustained release at a target that exceeds the anticipated daily dose (50 μg/day) required for protection against HIV, as benchmarked by the DPV intravaginal ring trial. The higher daily dose of DPV released by reservoir delivery systems likely explains pharmacokinetic differences between matrix and reservoir ARV-IUDs; indeed, reservoir delivery systems resulted in higher endometrial, vaginal secretion, and plasma concentrations of DPV. In order to extend the duration of release, a higher initial drug loading is a promising approach. The quantity of DPV loaded into these reservoirs (25.40 mg) was less than half of the initial API loaded into the Mirena IUD, for example, which contains 52 mg of levonorgestrel. Given that the drug loading assessed here already exceeded the saturation solubility of the core TPU, there is no reason to assume a higher mass fraction of drug would inhibit device fabrication or alter release kinetics. Other strategies, such as altering sheath polymer chemistry or thickness, could be explored to extend device duration; however, these would likely lower the daily DPV release rate. Ultimately, while this estimation of T50% potentiates device viability for up to a year, the actual duration of use must be rigorously evaluated in subsequent clinical trials.

Described herein is the successful development of both matrix and reservoir delivery systems for sustained release of multiclass, physicochemically distinct antiretrovirals in an integrated ARV-IUD design. Hydrophobic, highly crystalline TPU matrix can release RAL, MVC, and ETR for over one year while retaining 50% of its original drug, thus offering a consistent, theoretically protective dose of the triple drug combination. Moreover, a method for evaluating reservoir devices with low drug loading was demonstrated and showed that sheath thickness and sheath polymer chemistry play significant roles in mediating drug release. Ultimately, a candidate reservoir formulation for high DPV loading was selected and it exceeded the target duration of release and achieved and optimal daily release rate to meet the clinical specification. The in vivo performances of two delivery systems were tested (monolithic matrix and core-sheath reservoir) as candidates for long-acting delivery of a clinically relevant dose of the NNRTI dapivirine upon intrauterine instillation via an intrauterine device. It was shown that matrix and reservoir delivery systems can be effectively integrated with an IUD system, termed ARV-IUDs, and safely inserted trans-cervically into adult female baboons. ARV-IUDs were well tolerated with limited histological evidence of drug or delivery system-mediated adverse effects on local tissue. Intrauterine DPV delivery resulted in high concentrations of drug in the endometrium, cervix, and vagina, and both delivery systems resulted in high vaginal secretion DPV concentrations that measured approximately 300 to 1000-fold the IC90 of DPV in cervicovaginal fluid. Low systemic DPV concentrations were detected, suggesting that systemic circulation plays a small role in the transport of drug from the upper to lower FRT upon uterine delivery; this is the first evidence of a uterine to vaginal “first-pass” effect. Lastly, both matrix and reservoir delivery systems offered durable release of DPV in vivo suggestive of up to a year of drug release and protection against HIV in the vaginal tract.

This work is the first to exploit the IUD as a drug delivery modality for purposes beyond contraception. These findings represent a promising development towards long-acting multipurpose prevention technology and elucidates the potential of the uterus as an implantation site for drug delivery systems targeting both the upper and lower FRT. Given the tunable and modular design of our matrix and reservoir delivery systems ARV-IUDs, this work potentiates formulation of various prophylactic and therapeutic small molecules, alone and in combination, for long-acting delivery to the FRT. Once fully realized, this work has tremendous potential to motivate clinical translation of a novel long-acting MPT device which could alleviate the incidence unintended pregnancy and HIV among people with FRTs in regions where both public health challenges are endemic.

Thermoplastic polyurethanes (PY-PT42DE35 (“TPU1”), PY-PT83AE35 (“TPU2”), PY-PT72AE (“TPU3”), and PY-PT87AE (“TPU4”), dissolved overnight at 10% wt/vol in chloroform. Micronized Dapivirine (DPV) in polymer solution at 1% wt. drug/wt. polymer (wt/wt) or 16.6% wt/wt for ≥6 hours. Etravirine (ETR), Maraviroc (MVC) and Raltegravir (RAL) were dissolved in polymer solution at 1% wt. drug/wt. polymer (wt/wt) or 16.6% wt/wt for ≥6 hours. Polymer-drug solutions (40 mL) were poured into a 100 mm×15 mm glass petri dish and placed uncovered in a fume hood for 24 hours. Solvent cast films were placed in a high-vacuum desiccator for ≥4 hours on each side to remove residual solvent. Films were cut into disks with a 1-inch diameter die punch; disks were stacked and placed between two pieces of aluminum foil and compressed under a metal weight on a 120° C. dry bath. Thickness was monitored frequently until it measured 3.5 mm with digital calipers calibrated to 0.01 mm. Devices were cut from thick films using a custom rectangular 3.5 mm×13.3 mm die punch.

Reservoir delivery system prototypes were prepared using a heated coextrusion process. Briefly, core polymer (PY-PT43DE20) was melted and homogenized with the appropriate mass of dapivirine to achieve 1 wt % and 30 wt % core polymer loading. Core polymer and sheath polymer (PY-PT72AE or PY-PN73AE) were simultaneously extruded at 170-190° C. through a custom crosshead to meet target tube dimensions with a 1.5 mm inner diameter (lumen), 3.56 mm outer diameter, a 1.0 mm wall (core+sheath) thickness, and 100 to 200 μm sheath thickness. The inner crosshead diameter was varied mid-extrusion to generate devices with sheath thicknesses ranging from 100-200 μm while retaining a constant outer diameter. Material was cut to the desired length per device (13.33 mm) and in-spec devices were identified based on sheath thickness using brightfield microscopy. Devices were incubated for two weeks at 40° C. to equilibrate drug in the core and sheath compartments.

Matrix device prototype dimensions were measured using digital calipers. Reservoir device prototype height and outer diameter dimensions were measured using digital calipers; sheath thickness was measured using brightfield microscopy couples with image analysis.

High-performance liquid chromatography (HPLC) was used to measure DPV independently and RAL, MVC, and ETR as described.

For matrix and reservoir prototypes, drug content was extracted analyzed by HPLC.

The solubility of DPV, MVC, and ETR in candidate in vitro release media was determined at physiological temperature (37° C.).

Drug-free matrix devices of each TPU type in triplicate were used to assess swelling and equilibrium water content. Initial device length (L.) was measured using digital calipers and initial mass (M.) was recorded. Devices were immersed in PBS for 96 hours, then dried lightly to remove surface-associated liquid. Device length at equilibrium (L.) and mass at equilibrium (Me) were recorded.

In vitro drug release from matrix and reservoir delivery system prototypes was performed in a rotating shaker set to 60 RMP and 37° C. All release tests were performed in sink conditions in PBS supplemented with 2% w/v Cremophor® A25/Emulgin® B25. For the first 7-days of drug release testing, 1 mL samples were taken daily; following 7 days, samples were taken every 3-4 days. At all timepoints, bottles were drained and replaced with fresh media following sampling. For reservoir delivery system prototypes, three replicates of each formulation were end capped and one device was tested with no end cap to confirm the integrity of the end cap in preventing uncontrolled diffusion form the exposed core.

For matrix ARV-IUDs, A 21-gauge stainless steel needle was inserted longitudinally through a matrix device which was then rolled on a ceramic hot plate to form a hollowed, cylindrical monolithic matrix. Matrix devices were pushed onto the sheath of IUD frames and situated above the retrieval thread loop. Reservoirs were pushed onto the IUD frames, situated above the retrieval loops, and ends sealed with Loctite cyanoacrylate glue. Several studies have demonstrated the use of acrylate glue to prevent longitudinal diffusion from exposed reservoir cores in in vitro release studies. Polypropylene sutures were tied to the retrieval thread loop and frame arms were cut to measure approximately 14 mm across. All integrated ARV-IUDs underwent ethylene oxide sterilization prior to in vivo insertion.

Sterilized ARV-IUDs were loaded into a 12 Fr polypropylene insertion tube or medical grade stainless steel insertion tube measuring 4.4 mm to 5.15 mm outer diameter. Six reproductive aged (9-18 years old) female, parous Anubis baboons were used in a crossover study design. Prior to insertion, Misoprostol (400 μg) was administered to each animal to soften the cervix. Animals were sedated with ketamine, intubated endotracheally, and anesthetized with isoflurane. A speculum was inserted into the vaginal canal for visualization of the cervix and ARV-IUDs were implanted transcervically with ultrasound guidance. Devices were placed for 28 days and then removed with animals under the same sedation, intubation and atheization protocol as described for insertion. Following removal, females underwent a “wash-out” period of 5 weeks prior to placement of a second device constituting the second study arm.

Plasma samples were collected prior to ARV-IUD insertion, at hours 24, 48 and 72 after insertion, and weekly thereafter until removal. K2 EDTA-treaded tubes were used to collect 5 mL of plasma drawn from the medial saphenous vein at each timepoint. Tubes were centrifuged for 5 minutes at 2500 RCF, plasma was aspirated and deposited into a microcentrifuge tube. Secretion samples were collected prior to insertion and weekly thereafter until removal; vaginal secretions were obtained by insertion of Week-Cel cellulose sponges for 5 minutes into the vaginal tract. Vaginal biopsies were taken prior to insertion, at 7 days post-insertion, and upon device removal. Cervical biopsies were taken prior to insertion and upon device removal. Vaginal and cervical pinch biopsies were taken with a Kevorkian Biopsy device. Endometrial tissue was retrieved prior to insertion and immediately after IUD removal using a vacuum aspirator. All samples were stored at −80° C. until analysis.

Animals were monitored for signs of discomfort including decreased appetite, lethargy, and hunched posture. Tissue biopsies for histological analysis were fixed in 4% paraformaldehyde, dehydrated in alcohol, and embedded in paraffin. Cross sectional samples (5 μm) were taken and stained with hematoxylin and eosin (H&E) stain.

Stock solutions of dapivirine and its isotopically labeled internal standard (d-DPV) were prepared at 1 mg/mL in LC-MS-grade acetonitrile. We prepared working stocks containing unlabeled DPV in ACN at concentrations ranging from 30 to 22500 ng/mL. Calibration standards contained 10 ng/ml of internal standard and 0.1, 0.25, 0.5, 1, 2.5, 5, 10, and 50 ng/mL concentrations of unlabeled DPV. Quality control samples were prepared fresh for each sampling batch by spiking<1 μL of 22500 ng/ml DPV in ACN into neat or baseline biological samples before or after extraction to achieve final concentrations within the linear range of the standard curve.

Thawed plasma samples were precipitated with cold ACN, vortexed thoroughly, and centrifuged for 10 minutes at 10,000 RPM. Supernatant was filtered using a 0.22 μm PVDF syringe tip filter into a 200 μL glass autosampler vial. Solvent was removed by placing uncapped samples in a desiccator overnight. After solvent was completely evaporated, analytes were resuspended in an equal volume of ultrapure water and ACN containing internal standard (d-DPV). Tissue samples were screened for quality; samples with excess blood or negligible mass (<1 mg) were not included in analysis. Thawed tissue samples were precipitated with cold ACN containing internal standard (d₄-DPV) and homogenized in an Eppendorf tube containing 640 mg of 1.4 mm and six 2.8 mm zirconium oxide ceramic beads on a Precellys® 24 tissue homogenizer at 6,500 RPM for three cycles of 20 seconds. Samples were then centrifuged for 10 minutes at 10,000 RPM and supernatant filtered using a 0.22 μm PVDF syringe tip filter into a 200 μL glass autosampler vial containing an equal volume of ultrapure water. Vaginal secretion samples were thawed, diluted in 300 μL of phosphate buffered saline (PBS), and incubated for 1 hour at 4° C. Sponge and buffer were placed in Spin-X centrifuge tubes containing 0.45 μm cellulose acetate filters and precipitated with cold ACN containing internal standard (d₄-DPV), vortexed thoroughly, and centrifuged for 10 minutes at 10,000 RPM. Supernatant was filtered using a 0.22 μm PVDF syringe tip filter into a 200 μL glass autosampler vial containing an equal volume of ultrapure water. For quantification, the volume of vaginal secretion collected per samples was presumed to be 17.3 μL according to published data on vaginal mucosal volume collected by the Week-Cel method.

Delivery systems were removed from previously implanted ARV-IUDs and cut into three equal segments. Segments were massed and submerged in 10 mL of tetrahydrofuran (THF) in glass scintillation vials. Vials were sealed and incubated at 37° C. for at least 24 hours until the devices were fully dissolved. THE solutions were vortexed, diluted in acetonitrile (ACN), and filtered with a 0.22 μm syringe tip filter prior to drug content analysis by high-performance liquid chromatography (HPLC) using methods as described above.

Female baboons were treated with oral contraceptives (0.15 mg levonorgestrel and 0.03 mg ethinyl estradiol) daily throughout the study and beginning at least two weeks prior to initial IUD insertion to suppress cyclicity and maintain tissue homogeneity. Prior to all sampling timepoints, animals were sedated with ketamine; for tissue biopsy samples, biopsy areas were treated with silver nitrate to stop bleeding. The following medications were administered as needed for pain management throughout the study: Meloxicam (1.5 mg/mL), Buprenorphine (0.3 mg/mL), topical lidocaine (4%), Carprofen (50 mg/mL), and Ondansetron (2 mg/ml) for anti-nausea. Animal procedures were approved by the Institutional Animal Care and Use Committee (IACUC) at the Oregon National Primate Research Center (ONPRC). Animal husbandry was provided by ONPRC in accordance with the National Institutes of Health Guidelines for Care and Use of Laboratory Animals.

All data were presented as mean±standard deviation. Linear regressions and determinations of statistical significance were made using GraphPad Prism. Ordinary one-way ANOVAs multiple comparisons were used to calculate significant differences in drug loading, encapsulation efficiency, TPU swelling and equilibrium water content. Ordinary two-way ANOVA was used to calculate significant differences in prototype surface area to volume ratios with Dunnett's multiple comparisons test to compare matrix devices with reservoir devices. Comparison between drug concentrations were performed using Mann-Whitney rank comparison tests. Differences in residual drug analyses were assessed using parametric, unpaired t-tests. Significant differences were accepted when p value<0.05.

### Example 1: Design and Development of Matrix and Core-Sheath Reservoir Delivery Systems

Four TPUs with distinct attributes were selected for matrix delivery system investigation: PY-PT42DE35 (referred to herein as “TPU1”), PY-PT83AE35 (referred to herein as “TPU2”), PY-PT72AE (referred to herein as “TPU3”), and PYPT87AE (referred to herein as “TPU4”) as shown in Table 1.

TABLE 1
Summary of matrix delivery system prototype attributes.

| Matrix | | PU Classi- | Durometer | Water | Hydropho- |
|---|---|---|---|---|---|
| ID | TPU | fication | Hardness | Uptake | bicity |
| TPU1 | PY- | Aliphatic | 43D | 35% | Hydrophilic |
| | PT42DE35 | Polyether | | | |
| TPU2 | PY- | Aliphatic | 83A | 100% | Hydrophilic |
| | PT83AE35 | Polyether | | | |
| TPU3 | PY- | Aliphatic | 72D | <2% | Hydrophobic |
| | PT72AE | Polyether | | | |
| TPU4 | PY- | Aliphatic | 87A | <2% | Hydrophobic |
| | PT87AE | Polyether | | | |

The matrix delivery system prototype fabrication was tested to determine the relative water uptake and swelling behavior of the four TPU candidates. These metrics were predicted by measuring the equilibrium mass and dimensional change of drug-free matrix devices soaked in phosphate-buffered saline (PBS). As expected, TPU1 and TPU2 exhibited significantly greater swelling and equilibrium water content than hydrophobic TPU3 and TPU4. TPU2 exhibited significantly greater swelling and water uptake than TPU1; no differences were observed between TPU3 and TPU4. TPU matrix delivery system prototypes were prepared and loaded with 1 wt % (low loading) and 16.6 wt % (high loading) dapivirine (DPV), raltegravir (RAL), maraviroc (MVC) and etravirine (ETR). Only TPU4 was used for high drug loading prototypes. Matrix delivery system prototypes loaded with 1 wt % drug were translucent and showed no apparent drug crystallization. Those loaded with 16.6 wt % DPV, RAL, and ETR showed visible crystal formation and an opaque appearance; this is likely the result of drug being above the saturation solubility of the polymer. Four core-sheath reservoir delivery system configurations were fabricated to assess the effect of sheath polymer chemistry and sheath thickness on drug release as shown in Table 2. Reservoir devices loaded with 1 wt % DPV were translucent as the one or more active compounds (“drug”) was completely dissolved in the core polymer and had a hollow cylindrical structure.

TABLE 2
Summary of reservoir delivery prototype attributes.

| Reservoir | Sheath | Sheath | Sheath PU | Core | Core PU |
|---|---|---|---|---|---|
| ID | Thickness | Polymer | Description | Polymer | Description |
| R1 | 100 μm | PY- | Hydrophobic, mid | PY- | Hydrophilic, |
| R2 | 200 μm | PT72AE | HS/SS, polyether | PT43DE20 | high HS/SS*, |
| R3 | 100 μm | PY- | Hydrophobic, mid | | polyether |
| R4 | 200 μm | PN73AE | HS/SS, polycarbonate | | |

Upon validation of fabrication reproducibility, there were no significant differences found in the surface area to volume ratios of any TPU-type matrix device loaded with any ARV, nor were there any significant differences between matrix delivery systems and core-sheath reservoir delivery systems prototypes SA/V loaded with DPV. The relative standard deviation of SA/V for all devices was below 2.2, reflecting good uniformity within formulations. Both matrix delivery system and reservoir delivery system prototypes were successfully integrated into an IUD frame, demonstrating feasibility of both fabrication techniques at reproducibly generating devices amenable to system integration. The effective drug loading in each delivery system prototype was verified and calculated the encapsulation efficiency based on the theoretical drug loading. For all low loading matrix delivery system prototypes, the average effective loading is 0.91±0.14 wt % with an average encapsulation efficiency of 91.13±13.38%. Low loading TPU3-MVC, TPU4-MVC, and TPU4-ETR devices had significantly lower encapsulation efficiency than other TPU-ARV formulations. However, this difference is not reflected significantly in effective loading and therefore should not significantly affect drug release. High loading TPU4 matrix prototypes had an average effective loading of 16.46±1.12 w % with an encapsulation efficiency of 99.12±6.28% and no statistically significant differences among each ARV. Core-sheath reservoir delivery system prototypes were effectively loaded with 1.08±0.19% drug in the core which translates to an average encapsulation efficiency of 107.85±19.44%. No significant differences in effective drug loading or encapsulation efficiency were found between the four core-sheath reservoir delivery system groups.

FIGS. 6A-6C are graphs showing differential dapivirine release driven by polymer crystallinity and water penetration, in accordance with the present technology. The in vitro release kinetics of DPV from four TPU-based matrix delivery system prototypes were also investigated as shown in Table 1. Phosphate buffered saline with 2% w/v Cremophor, a nonionic surfactant, was used as dissolution media in order to enhance the solubility of DPV such that it could be detected in sink conditions above the lower limit of quantification via HPLC. It was determined that the saturation solubility of DPV in 2% Cremophor is 163.76±9.40 μg/mL. in vitro release of 1% DPV from TPU matrix devices revealed dependence on crystalline and water-uptake properties of each polymer.

FIG. 6A shows the percentage of swelling of each of the TPUs (TPU1, TPU2, TPU3, TPU4) without dapivirine. On the vertical axis is swelling in percentage. On the horizontal axis are each of the TPUs. The cumulative release rate for TPU1 and TPU2, two hydrophilic, water swelling polymers, were not statistically different over the 30-day release period. TPU3, a hydrophobic PU with a higher soft-to-hard segment ratio than the other three TPUs, resulted in the most rapid dissolution of DPV, releasing approximately 95% of encapsulated DPV by 30-days.

FIG. 6B is a graph of the cumulative release over time of DPV of each of the TPUs. All four TPUs released a DPV dose within a target 10-50 μg/day range. The release rate per day graph displays a sharp peak at early time points indicating burst release followed by a steady decline to near zero-order release by 30-days. A qualification for selecting an optimal TPU for DPV delivery in this application is the time to achieve 50% cumulative release of drug. This is based on the standard use time of hormonal IUDs, which in practice are removed once about half of the initial load of drug is depleted from the device. Here, this standard was used as a benchmark to evaluate the sustained release properties of candidate delivery system designs. Though the time to release 50% DPV (T_{50%}) is resolved within 30 days of in vitro release for three of the four TPUs assessed here, the cumulative release data was fit to empirical models of drug release in order to establish methods to predict T50% mathematically. The fit of first-order release kinetics to the data was assessed. Equations 1 and 2 describe drug release from a system where dissolution is dependent on a concentration gradient.

$$\frac{dC}{dt} = - kt \qquad Equation 1$$

$$Log ( {C}_{t} ) = Log ( {C}_{0} ) - \frac{k t}{2.303} \qquad Equation 2$$

Where k is the first order rate constant, Ct is the concentration of drug in solution at time t and C0 is the initial concentration of drug. Cumulative release data was also fit to the Higuchi model, which describes the release of drugs from an insoluble matrix based on Fickian diffusion and a square root of time relation given by the simplified expression:

$$Q = {k}_{h} {t}^{1 / 2} \qquad Equation 3$$

Where Q is the amount of drug release in time t and kₕ is the Higuchi release constant. Here, the square-root of time relation fits the in vitro release data better than first-order release kinetics for all TPU types as shown in Table 3.

TABLE 3
DPV matrix delivery system release parameters and T_{50%} estimation.

| | | Release Exponent | | Korsmeyer- | | |
|---|---|---|---|---|---|---|
| | | Higuchi: | First-Order: | Peppas | | |
| Loading | | k_{H} (%/h^{1/2}), | k (h⁻¹), | Constant: | Predicted | Actual |
| (wt %) | TPU | R² | R² | n, R² | T_{50%} (d) | T_{50%} (d) |
| 1% | TPU1 | 0.029, | 0.002, | 0.525, | 12 | 12 |
| | | 0.996 | 0.995 | 0.994 | | |
| | TPU2 | 0.027, | 0.002, | 0.549, | 13.5 | 12 |
| | | 0.996 | 0.992 | 0.996 | | |
| | TPU3 | 0.038, | 0.004, | 0.662, | 7 | 7 |
| | | 0.996 | 0.978 | 0.997 | | |
| | TPU4 | 0.017, | 0.0007, | 0.614, | 36 | — |
| | | 0.999 | 0.981 | 0.993 | | |
| 16.6% | TPU4 | 0.014, | 0.0005 | 0.674, | 53.5 | — |
| | | 0.995 | 0.964 | 0.976 | | |

Based on the best-fit square-root of time relation, T_{50%} was calculated for each TPU. Predicted values for T_{50%} closely matched experimentally observed data, indicating that this approach is suitable for predicting device use duration. Korsmeyer and Peppas provided a method for understanding drug release mechanisms of swelling and non-swelling polymeric systems, given by Equation 4:

$$\frac{{M}_{t}}{{M}_{∞}} = K {t}^{n} \qquad Equation 4$$

Where Mₜ/M_{∝} is the fraction of drug released at time t, K is the rate constant, and n is the release exponent. For a cylindrical polymeric delivery system which the rectangular prism delivery systems were approximated to be, the Korsmeyer-Peppas model defines 0.45<n<0.89 as anomalous transport characterized by both diffusion and matrix relaxation. The TPU matrices here have release exponents ranging from 0.525 to 0.614 which fall within the range of anomalous transport.

FIG. 6C is a graph of the DPV release per day in μg over time in hours. After screening the optimal TPU matrix delivery systems for DPV release, TPU4 was loaded with 16.6 wt % DPV. This loading was selected as it would allow a device to release at least 10 μg of DPV per day for at least one year while retaining at least 50% of its initial drug loading. It was found that at high loading in TPU4, DPV release in vitro was similar to that of the low loading condition. Indeed, the TPU4 matrix delivery systems released approximately 33.5% of loaded DPV after 30 days

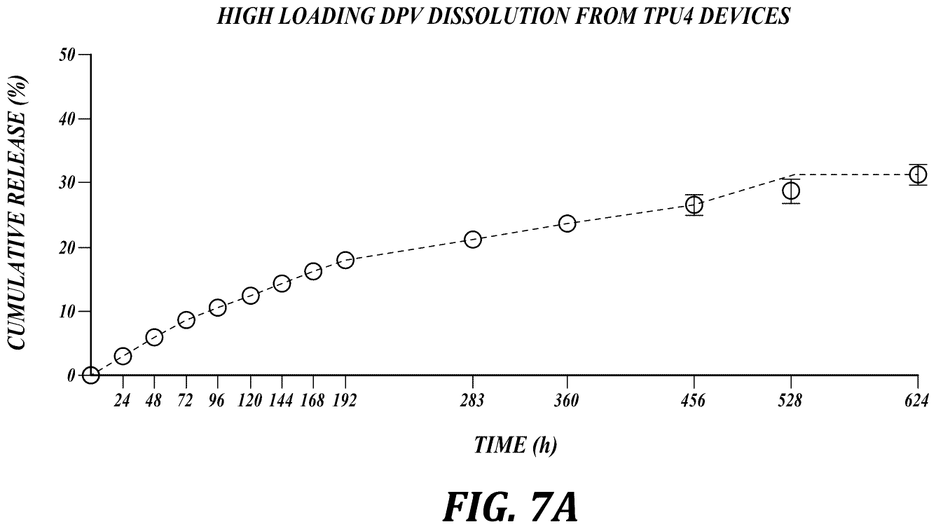

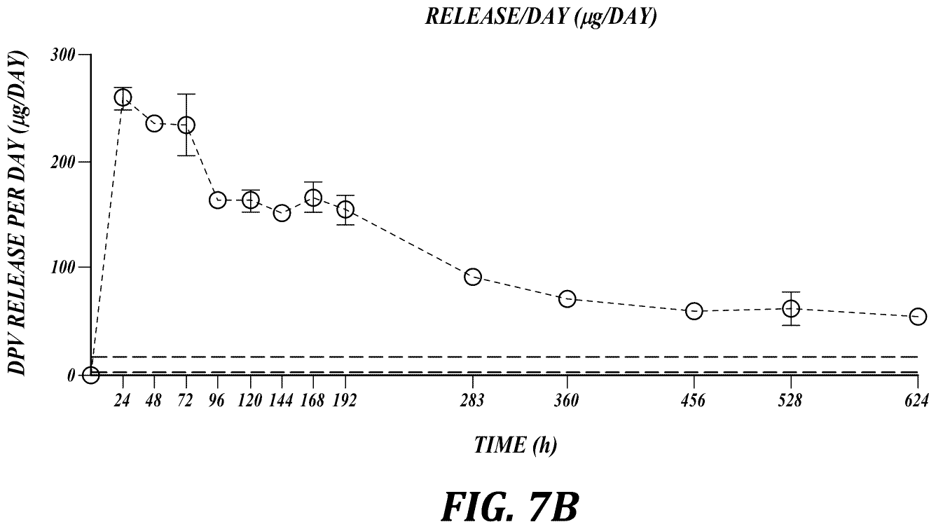

FIGS. 7A-7B are graphs of thermoplastic polyurethane (TPU4) highly loaded with dapivirine (DPV), in accordance with the present technology. A final release rate of approximately 62.56 μg/day (per each ⅓ segment of the device). First-order and Higuchi release models were fit to this data; here, the square-root of time dependence was most closely correlated with in vitro release data as shown in Table 1. The Higuchi model predicted that T50% would be reached at 53.5 days; this is significantly less than the one-year target, but greater than the T50% for 1% DPV in TPU4. The exponent of the Korsmeyer-Peppas model confirmed that anomalous release remains the driving drug release mechanism in this device (n=0.674).

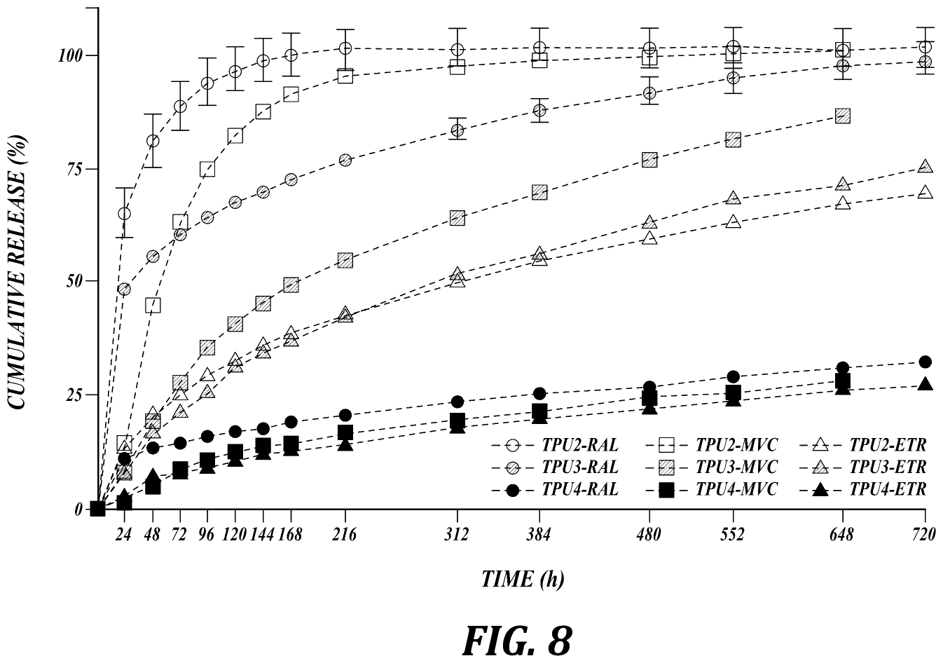

FIG. 8 is a graph showing 3-antiretroviral dissolution is dependent on active pharmaceutical ingredient and polymer properties, in accordance with the present technology. Three physiochemically distinct antiretrovirals—raltegravir (RAL), maraviroc (MVC), and etravirine (ETR)—were selected that represent a synergistic anti-HIV combination to probe the sensitivity of matrix release kinetics to active pharmaceutical ingredient properties. Screening the release of 1 wt % RAL, MVC and ETR from TPU2, TPU3 and TPU4 matrix delivery systems showed significantly variable release rates. TPU1 was excluded in this iteration.

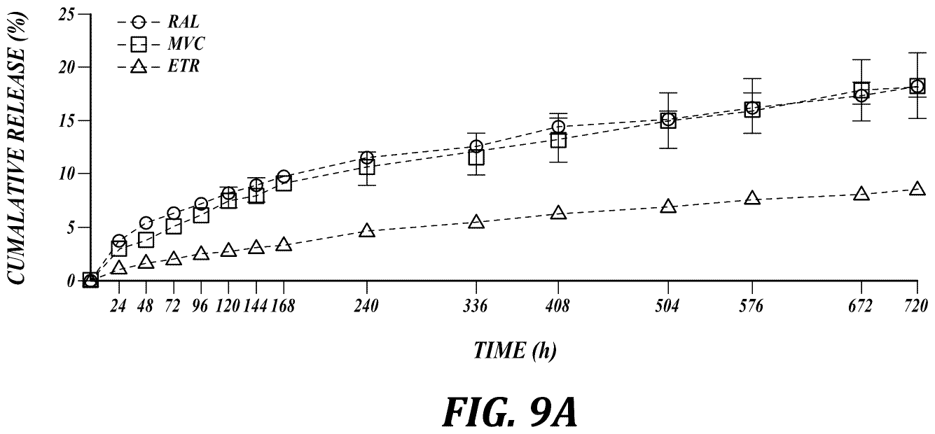

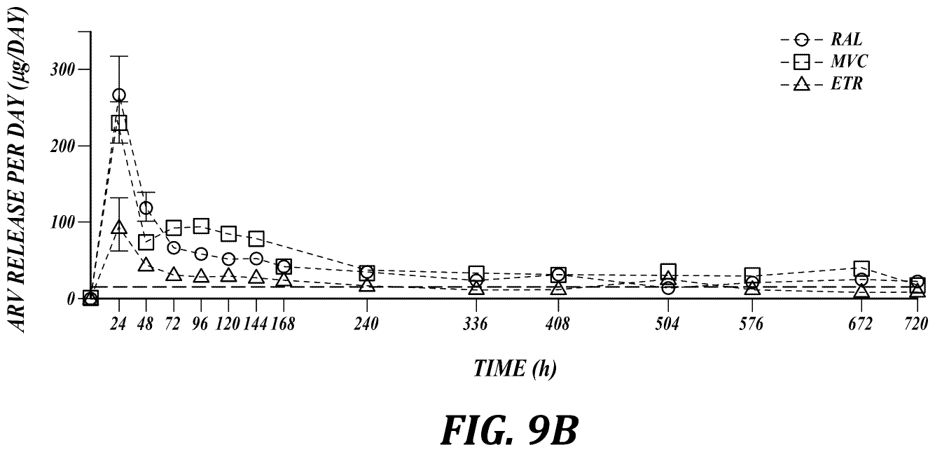

FIGS. 9A-9B are graphs of thermoplastic polyurethane highly loaded with raltegravir (RAL), etravirine (ETR), and maraviroc (MCV), in accordance with the present technology. The TPU2 matrix delivery systems released approximately 50% of encapsulated RAL and MVC within the first 24 hours of dissolution and reached 100% release by day 7 and 13, respectively. TPU3 matrix delivery systems also burst release approximately 50% of encapsulated RAL in the first 24 hours but then showed more sustained, first order release behavior with cumulative release reaching 100% at one month. TPU4 matrix delivery systems offered sustained and consistent dissolution of all ARVs, releasing 31.71% RAL, 33.83% MVC, and 27.70% ETR total by 30-days. Release within the target dose per day window was only achieved by TPU3 devices and TPU2 with ETR at 30 days (FIG. 8).

Release data fit to first order release kinetics, Higuchi release kinetics and the Korsmeyer-Peppas Model are summarized in Table 4.

TABLE 4
3-ARV matrix delivery system release parameters and T_{50%} estimation.

| | | | Release Exponent | | Korsmeyer- | | |
|---|---|---|---|---|---|---|---|
| | | | Higuchi: | First-Order: | Peppas | | |
| | Loading | | k_{H} (%/h^{1/2}), | k (h⁻¹), | Constant: | Predicted | Actual |
| ARV | (wt %) | TPU | R² | R² | n, R² | T_{50%} (d) | T_{50%} (d) |
| RAL | 1% | TPU2 | 0.052, | 0.016, | — | 2 | <1 |
| | | | 0.959 | 0.982 | | | |
| | | TPU3 | 0.023, | 0.004, | 0.201, | 7 | 1 |
| | | | 0.987 | 0.992 | 0.999 | | |
| | | TPU4 | 0.010, | 0.0004, | 0.333, | 109 | — |
| | | | 0.999 | 0.979 | 0.995 | | |
| | 16.6% | TPU4 | 0.006, | 0.0002, | 0.455, | 251 | — |
| | | | 0.998 | 0.970 | 0.999 | | |
| | 30% | TPU4 | 0.005, | 0.0002, | | 363 | — |
| | | | 0.998 | 0.98 | | | |
| MVC | 1% | TPU2 | 0.125, | 0.013, | — | 1 | 2.5 |
| | | | 0.986 | 0.984 | | | |
| | | TPU3 | 0.037, | 0.003, | 0.890, | 7.5 | 7 |
| | | | 0.978 | 0.992 | 0.960 | | |
| | | TPU4 | 0.012, | 0.0005, | 0.793, | 68 | — |
| | | | 0.973 | 0.928 | 0.886 | | |
| | 16.6% | TPU4 | 0.007, | 0.0002, | 0.529, | 221 | — |
| | | | 0.995 | 0.976 | 0.993 | | |
| | 30% | TPU4 | 0.003, | 0.0001, | | 737 | — |
| | | | 0.942 | 0.985 | | | |
| ETR | 1% | TPU2 | 0.026, | 0.002, | 0.484, | 15.5 | 13 |
| | | | 0.995 | 0.989 | 0.997 | | |
| | | TPU3 | 0.030, | 0.002, | 0.585, | 12 | 13 |
| | | | 0.997 | 0.995 | 0.995 | | |
| | | TPU4 | 0.011, | 0.0004, | 0.535, | 90 | — |
| | | | 0.999 | 0.975 | 0.998 | | |
| | 16.6% | TPU4 | 0.003, | 0.0001, | 0.599, | 857 | — |
| | | | 0.998 | 0.985 | 0.999 | | |

First order release was a good fit to Higuchi release for RAL in TPU2 and TPU3 and MVC in TPU3. First-order and Higuchi models are generally less able to predict T50% when it occurs upon burst release or at very early timepoints, such as the case of RAL in TPU2 (Higuchi) and TPU3 (first order) and MVC in TPU2. TPU4 offered the most favorable sustained release profiles for all ARVs as T50% was 109 days, 68 days, and 90 days for RAL, MVC, and ETR, respectively.

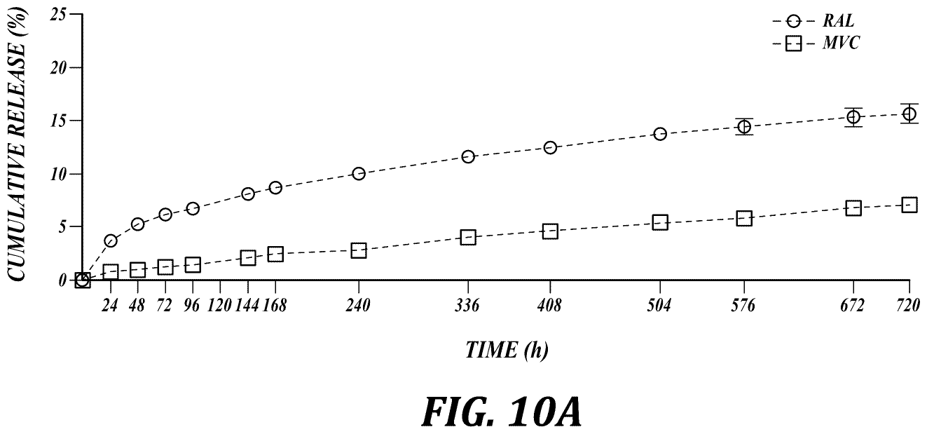

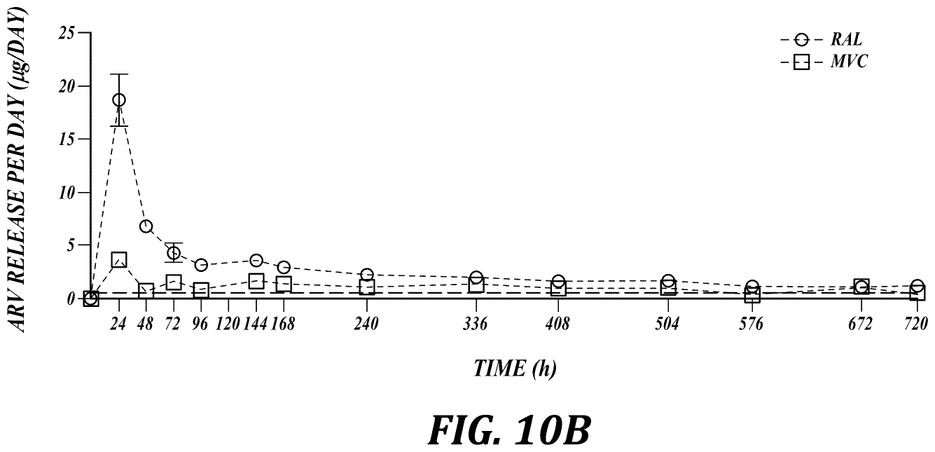

FIGS. 10A-10B are graphs of thermoplastic polyurethane highly loaded with raltegravir and maraviroc achieving one year of release or greater, in accordance with the present technology. Due to its durable release of all three ARVs at low loading, TPU4 was selected to investigate dissolution at high drug loading. RAL, MVC, and ETR were independently loaded at 16.6 wt % into TPU4 matrix delivery systems. Significant sustained release behavior of all ARVs from TPU4 matrix delivery systems was observed. After 30-days of in vitro dissolution, approximately 18% of RAL and MVC were released while only 8.5% of total ETR was released. The release rate of all three ARVs remained at or above the target dose per day for 30-days, leveling off around 22, 16, and 14 μg/day (per each ⅓ segment of the device) of RAL, MVC, and ETR, respectively. The Higuchi model most closely fit in vitro release data from all ARVs in comparison to first order release kinetics. The Korsmeyer-Peppas model indicates that anomalous, non-Fickian drug transport characterizes the matrix relaxation and diffusion-driven drug release mechanism present in these matrix delivery systems. Using the best-fit in vitro release model, it was predicted that T50% would be reached at 251, 221, and 857 days for RAL, MVC, and ETR, respectively. This represents a range from 0.60 to 2.34 years of sustained release with ETR exceeding our target of a one-year duration of action.

Acyclovir (ACV), a prophylactic agent used for the treatment and prevention of HSV-2, was loaded at 20 wt % into TPU4 matrix delivery systems. After 15-days of in vitro dissolution, 18% of ACV was released. ACV was consistently released above the calculated target release rate of 56 μg/day, with a minimum of 59 μg/day. ACV was consistently released above the calculated target release rate of 40 μg/day, with a minimum of 59 μg/day. Using the best-fit in vitro release model, it was predicted that T50% would be reached at 156 days.

The exact dosage of ACV necessary to suppress vaginal HSV replication is unknown. Here, the design constraints for effective acyclovir (ACV) release were based on previous literature with intravaginal rings. Thus, it was determined that a device with a minimum release rate of 40 μg/day may effectively protect against HSV-2.

Pritelivir release from reservoir and matrix devices for years-long durations was rapidly optimized. For core-sheath reservoirs, a single core TPU loaded at 1 wt %, with three sheath polymers of distinct hydrophobicity and crystallinity was investigated. Polymer extrusions were optimized to produce sheath thicknesses of 100 μM and 200 μM for a total of 6 reservoir prototypes. Daily release values from these protypes were highly dependent on both sheath polymer identity and thickness, spanning a range from 1-40 μg/day. These results demonstrate the tunability of pritelivir releasing reservoirs within the relevant target values. Matrix devices are also capable of meeting target specifications with a durable release rate of approximately 9% cumulative release after 20-days at 30 μg/day when formulated at a 20 wt % loading using a hydrophobic and crystalline TPU. Leveraging Higuchi release kinetic analysis, it was predicted that half of the original PTV dose to release from these 20 wt % matrix devices after approximately 500 days. Using a in-vitro-in-silico modelling workflow that leverages a high (20 wt %) and low (1 wt %) loading matrix device, it is predicted that 3 years of PTV release can be achieved with 30 wt % initial drug loading. In summary, the workflow can be applied to meet dissolution rates that span two-orders of magnitude across the matrix and reservoir DDS platforms.

In order to understand and more efficiently select drug loading targets for ARV release from matrix delivery system prototypes, in vitro release data was fit to empirical models of drug dissolution from monolithic solutions and monolithic dispersions. Drug release at early timepoints (Mₜ/M_{∝}<0.4) from a cylindrical matrix in which drug exists as a monolithic solution, meaning it is molecularly soluble and below the saturation solubility, can be described by Equation 5:

$$\frac{{M}_{t}}{{M}_{∞}} = 4 {( \frac{D t}{π {R}^{2}} )}^{1 / 2} - \frac{D t}{{R}^{2}} \qquad Equation 5$$

Where Mₜ/M_{∝} is the fraction of drug released at time t, D is the diffusion coefficient of drug in the polymer matrix, and R is the radius of the matrix. Drug release from a cylindrical monolithic dispersion at late timepoints (Mₜ/M_{∞} . . . >0.6) can be described by Equation 6:

$$\frac{{M}_{t}}{{M}_{∞}} = 1 - \frac{4}{2.40 {5}^{2}} exp ( - \frac{2.40 {5}^{2} D t}{{R}^{2}} ) \qquad Equation 6$$

Where Mₜ/M_{∞}, D, and R are as described above. The matrix delivery system prototypes can be approximated to a cylindrical device using the width as the dimensional equivalent of the diameter of a cylinder. The first seven consecutive timepoints of in vitro release data from TPU matrix delivery systems loaded with 1 wt % ARVs was fit to the appropriate dissolution equation (Equation 4 or 5) in order to determine the diffusion coefficient parameter for each ARV-TPU combination. Subsequently, the diffusion coefficient for each ARV-TPU combination was used to fit and predict dissolution from a saturated matrix delivery systems. Dissolution from a cylindrical monolithic dispersion is given by the implicit Equation 7:

$$\frac{{M}_{t}}{{M}_{∞}} + ( 1 - \frac{{M}_{t}}{{M}_{∞}} ) ln [ 1 - \frac{{M}_{t}}{{M}_{∞}} ] = \frac{4 D}{{R}^{2}} \cdot \frac{{c}_{s}}{{c}_{i n i}} \cdot t \qquad Equation 7$$

Where Mₜ/M_{∞}, D, and R are as defined above, cₛ is the saturation solubility of drug in the matrix polymer, and cᵢₙᵢ is the initial concentration (loading) of drug in the matrix. This equation is valid for all time. Using the diffusion coefficient for each ARV in TPU4 determined by their corresponding dissolution from a monolithic solution (Equation 4 or 5), 30-days of in vitro release data for TPU4 loaded with 16.6 wt % of each ARV was fit to Equation 6 for monolithic dispersions. This allowed us to determine cₛ for each drug in TPU4, thereby fully parametrizing the dissolution equation for a monolithic dispersion (i.e., a matrix delivery system).

The saturation solubility of each ARV in TPU4 derived from Equation 9 was used as an approximation for the saturation solubility in the remaining TPUs tested for each ARV in order to assess the ability of any drug-polymer dispersion to achieve at least one year of sustained drug release. The diffusion coefficient was derived from Equation 4 or 5 for each TPU-ARV combination. The fraction of drug released at a constant time, one year, as a function of drug loading was plotted. It was then determined which TPUs could release 50% or less of encapsulated drug in the target time period. These curves represented drug dissolution from a monolithic dispersion; therefore, a range from 5 to 50 wt % was plotted, the lower bound representing an approximation of the saturation solubility of the ARVs and the upper bound indicating the highest loading that we assume would be practically feasible to manufacture. It was found that no TPU is capable of releasing DPV for one year at any loading. Only TPU4 can offer sustained release of RAL and MVC for one year, requiring 25.21 wt % and 24.28 wt % of each ARV, respectively. All TPUs tested for ETR are able to deliver the drug for at least one year with loading within the target window—TPU2 at 49.40 wt % ETR, TPU3 at 42.47 wt % and TPU4 at 5.22 wt %.

Subsequently, the release of each ARV from TPU4 was modeled for one, two, and three years to determine the required drug loading to achieve an extended duration of use. This model confirmed that no drug loading up to the theoretical 50 wt % maximum is capable of offering one year of release of DPV. The requisite loading for one year of sustained release of RAL, MVC, and ETR agree with those demonstrated in the previous model. RAL and MVC can achieve two years of sustained release if loaded at 50.34 wt % and 48.62 wt %, respectively, with RAL slightly exceeding the maximum theoretical loading of 50 wt %. Neither RAL nor MVC can reach three years of sustained release within the loading maximum defined here. ETR loaded at approximately 10.46 wt % and 15.70 wt % can achieve two and three years of release, respectively.

In accordance with the in-silico prediction of ARV release from cylindrical monolithic dispersions, a very highly-loaded TPU4 matrix delivery system containing over 25 wt % RAL and MVC (approximately 30 wt %) was fabricated to optimize the probability of predicting T_{50%}>1 year. In vitro dissolution testing was conducted for 30 days and slower cumulative release rates than the 16.6 wt % loading RAL and MVC TPU4 delivery systems was observed. Indeed, 30 wt % TPU4-RAL release approximately 17% of encapsulated drug at 30 days, while 30 wt % TPU4-MVC released even less, approximately 7% of encapsulated drug (FIG. 5E). The release rate of RAL and MVC remained above the target dose per day by study terminus at 41.13 μg/day and 27.65 μg/day, respectively (per each ⅓ segment of the device). The release of very highly loaded MVC was best described best mathematically by the first-order equation (whereas RAL dissolution was best described by the Higuchi, square-root of time relation (Supplementary FIG. 8B), as presented in Table 4. The best-fit release exponents were used to predict T50% for RAL and MVC, which was 363 days and 737 days, respectively.

### Example 2: In Vitro Testing and in Silico Modeling of Single ARV Core-Sheath Reservoir Delivery System

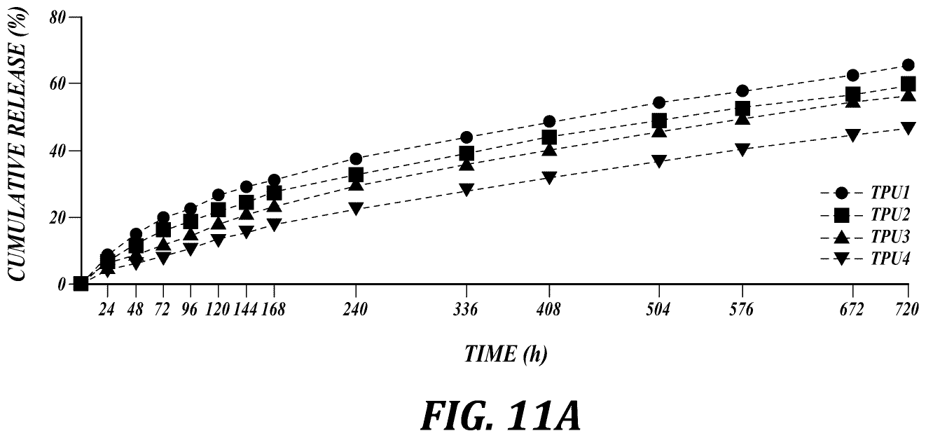

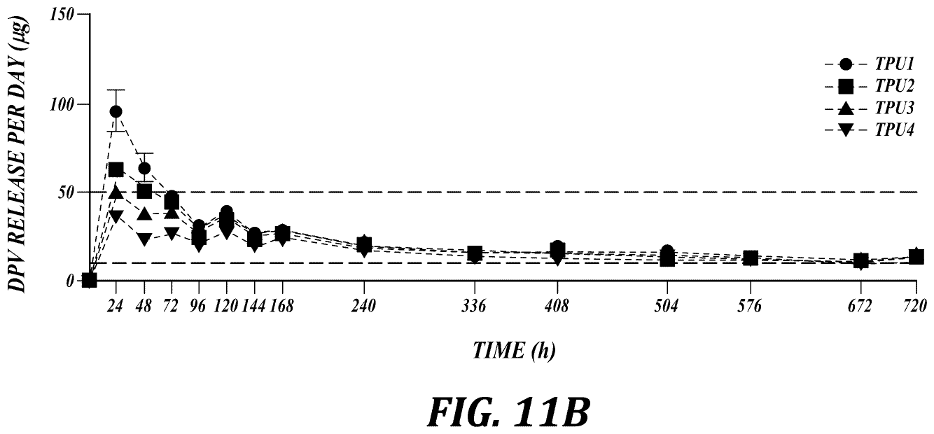

FIGS. 11A-11B are graphs showing in vitro release of an example delivery system loaded with 1 wt % of dapivirine, in accordance with the present technology;

Four designs of core-sheath reservoir delivery systems for sustained release of DPV as shown in Table 2. The groups included two sheath polymer chemistries combined with two sheath membrane thicknesses.

The in vitro release profile of DPV loaded at 1 wt % from the reservoir formulations corresponded with predicted relative release rates based on sheath thickness: reservoirs with 100 μm sheaths (R1 & R3) released drug faster than 200 μm reservoir devices with the same respective sheath polymers as shown in FIG. 11A. Both polyether sheath reservoirs (R1 & R2) released DPV faster than polycarbonate sheath reservoirs (R3 & R4). All reservoir formulations released DPV within the target dose of 10-50 μg/day, ranging from 11.7 μg/day to 15.7 μg/day at 30 days. In order to understand the drug release kinetics of these low-loading reservoir devices, it was determined the saturation solubility of DPV in the core polymer to determine if these reservoirs contained a constant or a non-constant activity source. It was found that the saturation solubility of DPV in a PY-PT43DE20 core is 5.46±0.93 wt %. Thus, with DPV loaded at 1 wt % which is below the saturation solubility, these reservoirs contain a nonconstant activity source. Pursuant to the behavior of reservoirs with a nonconstant activity source, these reservoirs released drug following first-order release kinetics. Despite this expectation, drug release from all four reservoir devices was described slightly better by a square-root of time dependence. Similar to the matrix delivery systems, the best-fit release profile, here the square-root of time dependence, was used to calculate the predicted T50% of each reservoir as shown in Table 5. These predictions were generally less accurate than those made for the matrix delivery systems, with an average relative error of 19.7%.

TABLE 5
DPV reservoir delivery system release
parameters and T_{50%} estimation

| | | | | Pre- | |
|---|---|---|---|---|---|
| Loading | | Release Exponent | | dicted | Actual |
| (% | Reser- | Higuchi: | First-Order: | T_{50%} | T_{50%} |
| w/w) | voir | k_{H} (%/h^{1/2}), R² | k (h⁻¹), R² | (d) | (d) |
| 1% | R1 | 0.025, 0.998 | 0.001, 0.993 | 16 | 19 |
| | R2 | 0.024, 0.999 | 0.001, 0.992 | 18 | 22 |
| | R3 | 0.024, 0.999 | 0.001, 0.996 | 18 | 24 |
| | R4 | 0.020, 0.997 | 0.0008, 0.997 | 26 | >30 |

In order to obtain more accurate predictions of reservoir delivery system behavior and design high-loading reservoirs capable of releasing drug for at least one-year, in vitro release data was fit to empirical equations for drug dissolution from reservoirs with constant and non-constant activity sources. The equation for drug dissolution from a core-sheath reservoir with a nonconstant activity source, meaning that drug exists in the core polymer below its saturation solubility, is given by Equation 8:

$$\frac{{M}_{t}}{{M}_{∞}} = 1 - exp [ - \frac{( {R}_{i} H + {R}_{o} H + 2 {R}_{i} {R}_{o} ) D K t}{{R}_{i}^{2} H ( {R}_{o} - {R}_{i} )} ] \qquad Equation 8$$

Where Mₜ/M_{∞} is the fraction of drug released at time t, Ri is the inner radius (radius of the core), Rₒ is the outer radius (radius of the core+sheath), H is the height, D is the diffusion coefficient of drug through the sheath, and K is the partition coefficient of drug between the core and sheath. Equation 8 was fit to the first 30 days in in vitro release of DPV from each of the four reservoir delivery system prototypes in order to determine the product of the diffusion and partitioning coefficient, given by the parameter DK.

With DK known for each formulation and the experimentally determined value for the saturation solubility of DPV in the core polymer allowed us to parameterize the expression for drug release from a reservoir with a constant activity source, given by Equation 9:

$${M}_{c} = \frac{2 π H D K {c}_{s}}{ln ( \frac{{R}_{o}}{{R}_{i}} )} \cdot t \qquad Equation 9$$

Where Mₜ represents the amount of drug released at time t, cs represents the saturation solubility of drug in the core polymer, and Ri, Ro, H, D, and K are as defined above. Using Equation 9, the release of DPV from each reservoir delivery system prototype was used to determine the final mass of drug released at one year. As expected, a reservoir with a constant activity source, in which drug is loaded above the saturation solubility of the core polymer, gives drug release with zero-order release behavior. From these predictions the mass and loading of DPV required to make one-year equal T50% for each reservoir was determined, taken as double the total amount of drug released in one year as shown in Table 6. the dose of drug released per day by each reservoir was also calculated, given by the slope of each release prediction.

TABLE 6
Predicted loading and release rate for highly-
loaded reservoir delivery system prototypes.

| Reservoir | Total DPV | | | |
|---|---|---|---|---|
| delivery | release in one | Target DPV | Core loading | Daily release |
| system | year (mg) | loading (mg) * | (% wt) ** | rate (μg/day) |
| R1 | 29.00 | 58.00 | 52.76% | 79.2 |
| R2 | 19.32 | 38.64 | 35.15% | 52.8 |
| R3 | 22.2 | 44.40 | 47.41% | 60.0 |
| R4 | 12.34 | 24.68 | 26.35% | 33.6 |

Informed by the predictions of DPV release from TPU reservoirs loaded above the core polymer saturation solubility, the leading candidate reservoir formulation, R4, was loaded with 30 wt % DPV in the core polymer. This loading was selected as it approximates that of the predicted 26.35 wt % to achieve one year of DPV release at 33.6 μg/day (Table 6). Also included was a comparator reservoir formulation, R3, loaded with the same wt % DPV in the core. In vitro release was conducted for 30 days in sink conditions and minimal difference in cumulative release was observed between the two formulations. The equilibrium dosage fell well within the target dose per day with R3 (full device) releasing an average of 24.23 (±3.79) μg/day for and R4 (full device) releasing 22.08 (±3.75) μg/day at day 30. Over the entire 30-day period, R3 released an average dose of 51.76 μg/day and R4 an average dose of 41.66 μg/day; these dosages are similar to those estimated by our predictions indicated in Table 7, where R3 was expected to release 60.0 μg/day and R4 33.6 μg/day given a core loaded with a constant activity source.

TABLE 7
Estimated duration of use of highly loaded DPV reservoirs.

| Reservoir | | | Cumulative | |
|---|---|---|---|---|
| delivery | Zero-Order | | release (%) | T_{50%} |
| system | Slope | R² | at 1 year | (days) |
| R3 | 0.0049 | 0.961 | 43.07 | 424.45 |
| | (0.0094) | (0.987) | (88.16) | (218.52) |
| R4 | 0.0047 | 0.987 | 41.90 | 436.25 |
| | (0.0084) | (0.990) | (73.66) | (247.10) |

The modelling predictions were further validated by fitting a zero-order line through the full 30-day IVR dataset for each reservoir and predicting the time it would take for each device to reach T50% (Table 7). It was found that R3 would take approximately 424 days to reach T50% and would release 43.07% of its initial cargo in one year, while R4 would take 436 days to reach T50% and would release only 41.90% of its cargo in one year.

### Example 3: ARV Delivery System Integration, Insertion, and Placement in Baboon Uteri

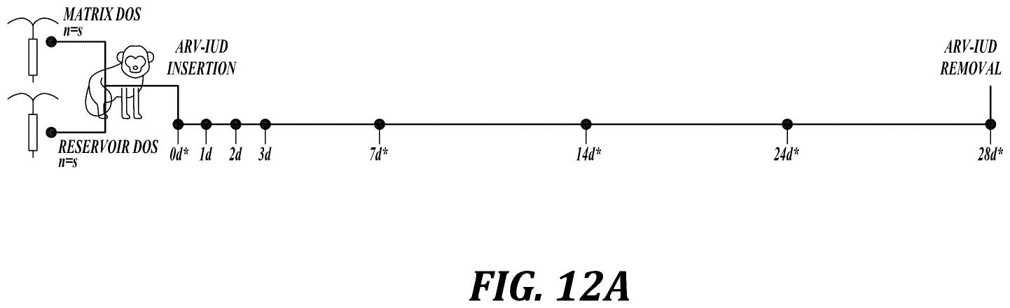

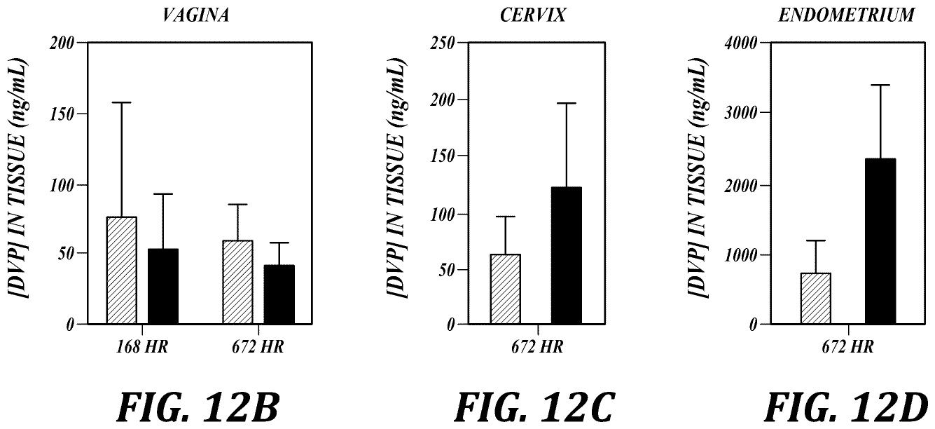

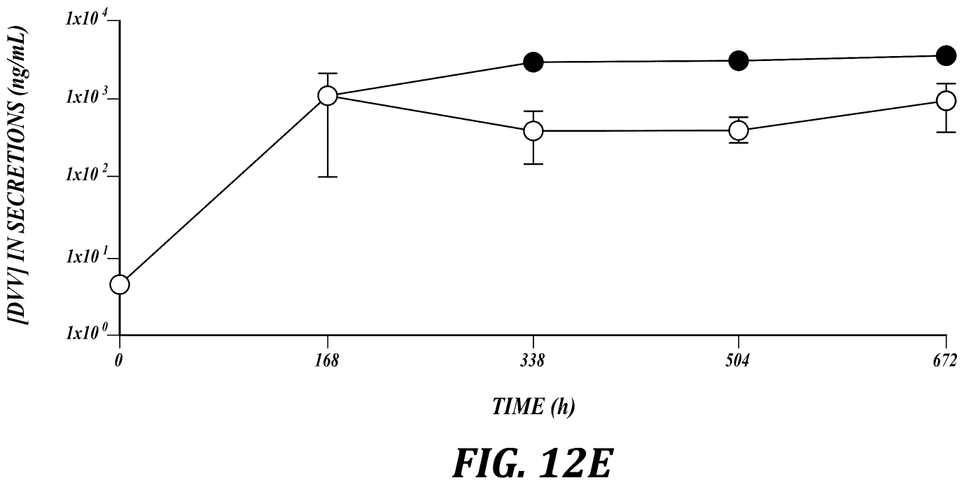

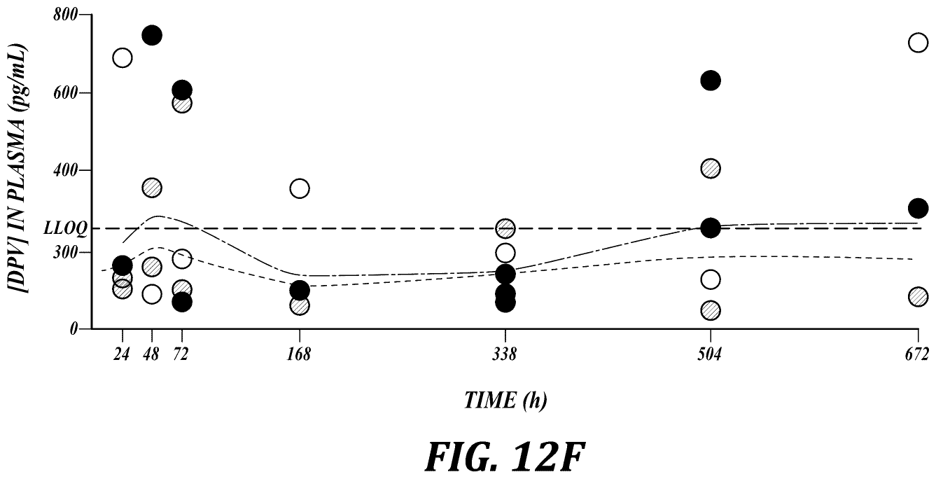

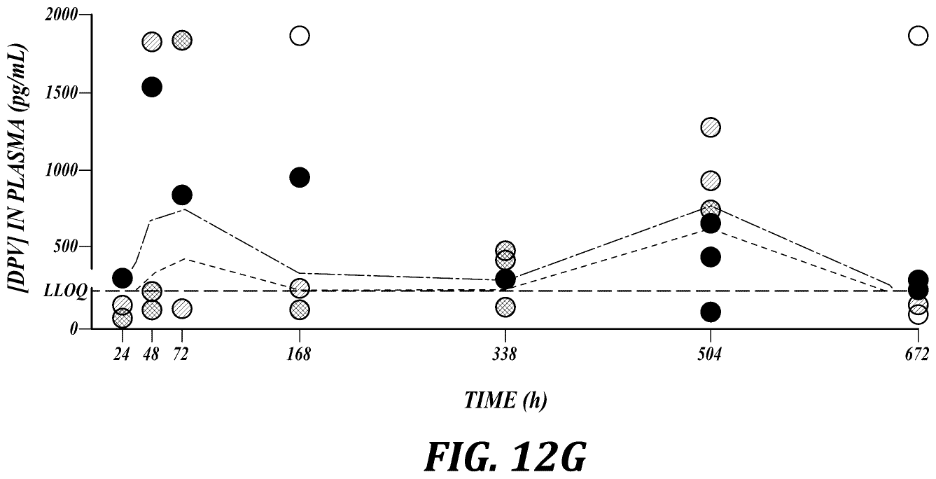

FIGS. 12A-12F are graphs illustrating dapivirine concentrations in tissue, secretions, and plasma of an example intrauterine device inserted into a baboon uteri. In order to investigate the pharmacokinetics of ARV delivered to the intrauterine space, matrix and core-sheath reservoir delivery systems were incorporated onto an intrauterine frame. The delivery systems were situated on the vertical body of the IUD frame such that insertion into baboon uteri with standard polypropylene or custom stainless steel 12-14 French insertion tubes was feasible. Placement of the ARV-IUDs was confirmed via fluoroscopy and ultrasonography. Indeed, ARV-IUDs were situated evenly within the uterine cavity with the body containing delivery systems positioned superior to the cervical canal.

Histological assessment of vaginal, cervical, and endometrial tissue after one month of ARV-IUD instillation indicated minimal impact of this device on tissue microenvironment. Vaginal tissue biopsies taken pre-insertion and 28-days post-insertion show characteristic features of healthy a vaginal epithelium, including a multilayered stratified squamous epithelium consisting of enucleated cells, a densely nucleated basal cell layer, and an inconspicuous lamina propria. Vaginal tissue biopsied 28-days post insertion has an intact stratified squamous epithelium that measures approximately 200 μm thick. Both pre-insertion and 28-days post-insertion cervical biopsies contain regions of a single columnar epithelium, while the underlying cervical stroma is composed of densely vascularized, fibromuscular tissue. The 28-day post-insertion cervical biopsy contains a thick stratus corneum of the exocervix; the epithelium overlays a prominent lymphoid aggregate, a feature of mucosa-associated lymphoid tissue (MALT) in the type I mucosa of the endocervix. Some cellular atypia is notable as a few epithelial cells display irregular nuclear membranes and perinuclear halos. The pre-insertion endometrium consists of a columnar epithelium that forms tubular glands overlaying a thick vascular stroma. An increase in mononuclear cells is notable throughout the stroma of the 28-day post-insertion endometrial biopsy along with some epithelial erosion; this is indicative of an inflammatory response and contact with a foreign body.

In a head-to-head pharmacokinetic study, matrix and core-sheath reservoir ARV-IUDs were inserted into baboons (n=6) and DPV concentrations in vaginal, cervical, and endometrial tissue, vaginal secretions, and plasma were quatified over a 28-day time course, as shown in FIG. 12A. No quantifiable drug was measured in baseline tissue samples taken 1 hour to 1 week prior to IUD insertion. One-week post-insertion of ARV-IUDs, vaginal tissue samples measured an average DPV concentration of 78.1 (±80.5) ng/ml; this value declined slightly but not statistically significantly to 61.1 (±25.6) ng/ml at device removal as shown in FIG. 12B and Table 8.

TABLE 8
DPV concentrations in baboon FRT tissue.

| | Time | [DPV] (ng/mL) (SD) | | Significance |
|---|---|---|---|---|
| Tissue Type | (hr) | Matrix | Reservoir | (p-value) |
| Vaginal | 168 | 78.06 (80.5) | 54.5 (39.0) | NS (0.841) |
| | 672 | 61.1 (25.6) | 43.16 (14.94) | NS (0.343) |
| Cervical | 672 | 65.6 (31.5) | 124.56 (72.48) | NS (0.132) |
| Endometrial | 672 | 759.7 (443.2) | 2380.0 (1028) | ** (0.004) |

By comparison, reservoir ARV-IUDs resulted in vaginal tissue samples with an average DPV concentration of 54.5 (±39.0) ng/mL at one week. In the cervix, matrix ARV-IUDs resulted in an average DPV concentration of 65.6 (±31.5) ng/ml at device removal, while reservoir ARV-IUDs resulted in 124.6 (±72.5) ng/ml of DPV in the cervix as shown in FIG. 12C. Endometrial DPV concentrations were high for both matrix and reservoir ARV-IUDs at device removal, measuring 759.7 (±443.2) ng/ml and 2380.0 (±1028) ng/ml, respectively (FIG. 8D, Table 8). The highest measured endometrial DPV concentration remains nearly 100-fold below the maximum tolerable DPV concentration tested on cervicovaginal tissue (0.33 mg/mL). The differences in tissue DPV concentrations for all biopsy locations were not statistically significant between matrix and reservoir delivery systems. For all biopsy locations, it was confirmed that tissue concentrations returned to baseline prior to reinsertion of devices in the second study arm.

DPV concentrations were measured in vaginal secretions weekly after insertion of matrix and reservoir ARV-IUDs as shown in FIG. 12E. Matrix ARV-IUDs resulted high vaginal secretion concentrations 7-days post-insertion, averaging 1073.24 (±1376.87) ng/ml. This concentration was durable out to 28-days, where DPV measured 933.68 (±799.76) ng/mL. Similarly, reservoir delivery system ARV-IUDs resulted in DPV concentrations of 1037.44 (±46.41) ng/mL and 3536.51 ng/mL in vaginal secretions at day 7 and day 28, respectively. At IUD removal, matrix and reservoir delivery system ARV-IUDs resulted in vaginal secretion concentrations were approximately 283 times and 1072 times the IC90 determined in ex vivo cervicovaginal tissue culture (3.3 ng/ml).

For matrix delivery system ARV-IUDs, a quantifiable plasma DPV concentration was measured at the first sampled timepoint, 24 hours after IUD insertion, in only one of six baboons (658.71 pg/mL) as shown in FIG. 12F. The remaining five animals had individual plasma DPV concentrations near or below the lower limit of quantification (LLOQ) for this assay (250 pg/mL), collectively averaging a plasma DPV concentration of 137.53 pg/mL. Thereafter throughout the 28-day study period, each baboon exhibited two to three DPV plasma concentrations that were above the LLOQ, ranging from 304.14-749.11 ng/ml with an average Cₘₐₓ of 539.4 (±221.1) pg/mL. Within this range of values, the individual tmax per animal occurred at hours 48, 72, 336, 504 and 672 equating to a median tmax of 420 hours (17 days). While all plasma samples measured a DPV concentration were above the lower limit of detection (LLOD), suggesting that they do contain some concentration of DPV, the arithmetic and geometric means of aggregated plasma DPV concentrations for the duration of the study are consistently below or very close to the LLOQ. Reservoir delivery system ARV-IUDs resulted in more significant, quantifiable plasma concentrations of DPV as shown in FIG. 12G. Plasma DPV concentrations increased from insertion to day 3, reaching a peak of 744.75 (±698.89) pg/mL three-days post-insertion (tmax). A trough at 14 days post-insertion, which measured 295.37.78 (±147.16) pg/mL, was followed by another peak in concentration at 21 days post-insertion of 768.36 (±389.98) pg/mL. Upon device removal, the average DPV concentration in plasma measured 192.30 (±88.43) pg/mL. Reservoir ARV-IUDs resulted in a more consistent pattern of DPV distribution in systemic circulation compared to matrix ARV-IUDs; however, neither delivery system resulted in plasma DPV concentrations that were comparable in magnitude to those measured in LFRT tissue or secretions.

The amount of residual DPV in each delivery system was quantified upon 28 days of in utero instillation order to predict the duration of action of matrix and reservoir delivery systems. Matrix ARV-IUDs retained an average of 81.78% (±5.44%) of their initially loaded DPV; thus, matrix delivery systems released approximately 18% of their DPV after 28 days in utero as shown in Table 9.

TABLE 9
Residual drug analysis from removed ARV-IUD delivery systems.

| | Initial DPV | | | |
|---|---|---|---|---|
| Delivery | loading | Total DPV | Average dose | T_{50%} |
| System | (mg) (wt %) | released (mg) | (μg/day) | (days) |
| Matrix | 12.3 (16.0) | 2.67 | 80.97 | 76.85 |
| Reservoir | 25.4 (18.8) | 2.94 | 101.75 | 303.58 |

By comparison, reservoir ARV-IUDs retained on average 95.84% (±2.93%) of initial DPV and released approximately 4.2% of their initial drug load after 28 days. The difference in percent of drug released between matrix and reservoir delivery system was statistically significant (p=0.0002). Assuming a constant release rate, matrix delivery systems had a daily release rate of 80.97 (±29.28) μg/day, while reservoir delivery systems released approximately 101.75 (±60.23) μg/day of DPV (Table 9); this difference in daily dose was not significant (p=0.4648). Upon extrapolation, matrix delivery systems would release 50% of their total drug loading after 76.85 days of use (T50%); reservoir delivery systems would reach T50% after 303.58 days.

### Example 4: Triple Active Compound Delivery System In Vivo Study

Described herein is a study conducted on an intrauterine system having a plurality of delivery systems (such as shown in FIGS. 1D and 1E). This study included three matrix delivery systems. The first delivery system included raltegravir (RAL), the second delivery system included etravirine (ETR), and the third delivery system included maraviroc (MVC). The intrauterine system was implanted and retained in the uterus of a patient for 28 days.

Multiple samples were taken before, during, and after insertion of the intrauterine system. Plasma samples were taken an hour before insertion, at insertion, 24 hours after insertion, 48 hours after insertion, 72 hours after insertion, 7 days after insertion, 14 days after insertion, 21 days after insertion, and 28 days after insertion (when the intrauterine system was removed). Vaginal swabs were taken an hour before insertion, at 7 days after insertion, 14 days after insertion, 21 days after insertion, and 28 days after insertion. Vaginal biopsies were taken an hour before insertion, 7 days after insertion, and 28 days after insertion. Endometrial and cervical biopsies were taken an hour before insertion and 28 days after insertion. Histology was also performed an hour before insertion, and consistent visual inspection was done throughout for safety.

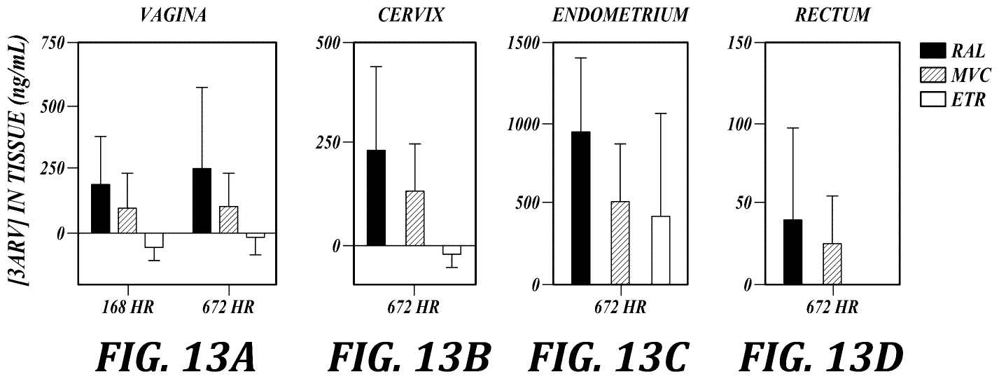

FIGS. 13A-13D are graphs of tissue concentrations of raltegravir (RAL), etravirine (ETR), and maraviroc (MVC), in the lower female reproductive tract (LFRT) and the rectum, in accordance with the present technology. On the vertical axis is the three ARV (RAL, ETS, MVC) in the tissue in ng/ml. On the horizontal axis is the time at 168 hours and/or 672 hours. The data found from these samples is summarized in Table 10.

TABLE 10
Tissue concentrations of RAL, MVC,
and ETR in the FRT & Rectum

| | | [RAL] | [MVC] | [ETR] |
|---|---|---|---|---|
| | | ng/mL | ng/mL | ng/mL |
| Vagina | 7 days | 193.79 | 103.79 | — |
| | 28 days | 253.3 | 108.30 | — |
| Cervix | 28 days | 237.26 | 137.275 | — |
| Endometrium | 28 days | 237.26 | 523.41 | 434.47 |
| Rectum | 28 days | 39.93 | 25.72 | — |

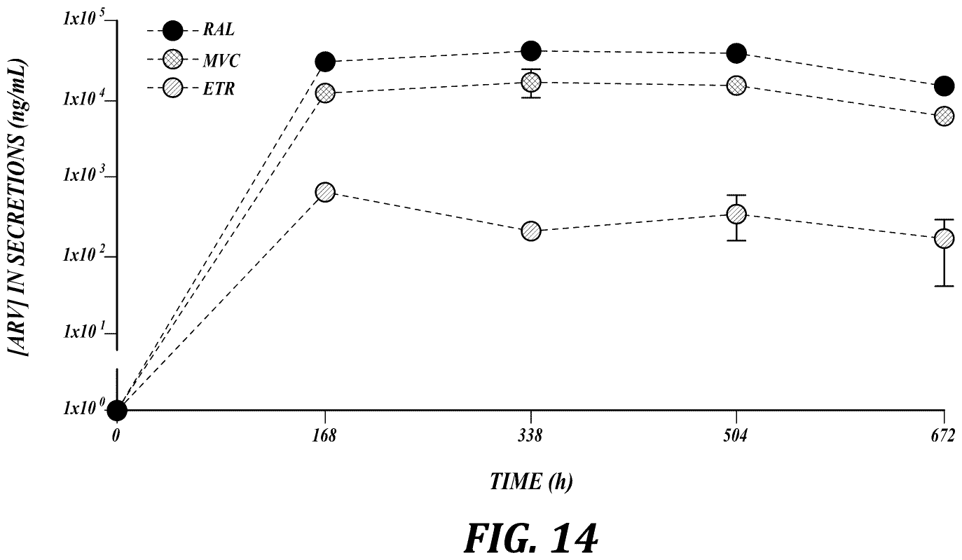

FIG. 14 is a graph showing the concentration of anti-retroviral pharmaceutical compositions in vaginal secretions, in accordance with the present technology. On the vertical axis is the [ARV] in secretions in ng/ml. On the horizontal axis is the time in hours. Shown is the time at insertion, 168 hours after insertion, 336 hours after insertion, 504 hours after insertion, and 672 hours after insertion. Table 11 should the mean and standard deviation of each ARV.

TABLE 11
Mean and standard deviation of RAL,
MVC, and ETR in vaginal secretions

| | [RAL] ng/mL | [MVC] ng/mL | [ETR] ng/mL |
|---|---|---|---|
| Mean | 32902.07 | 12752.31 | 344.30 |
| St. Dev. | 13349.60 | 5186.16 | 219.40 |

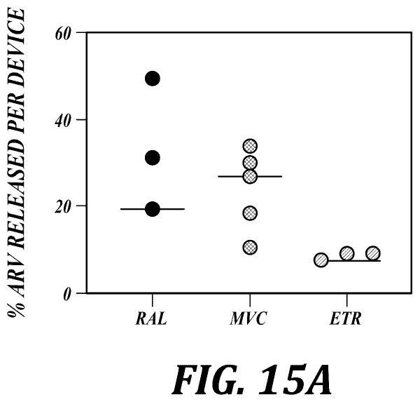

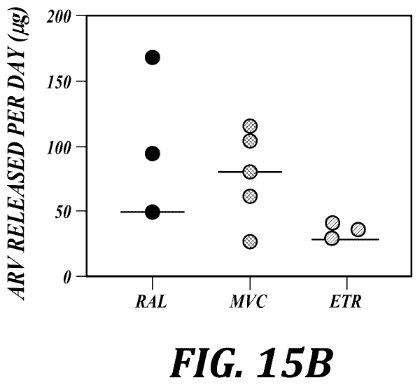

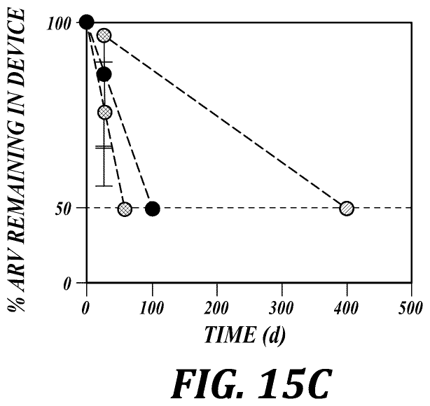

FIGS. 15A-15C are graphs showing post-use assays of residual anti-retroviral pharmaceutical compositions in removed intrauterine devices, in accordance with the present technology. FIG. 15A shows the percentage of ARV released per device for each of the three ARVs (RAL, MVC, ETR). FIG. 15B shows the amount of ARV released per day in micrograms for each of the three ARVs. FIG. 15C shows the percentage of ARV remaining in each device over time, in days. Table 12 is a summary of the results found in FIGS. 15A-15C.

TABLE 12
Post-use assay of residual ARVs in removed IUDS.

| | Initial DPV loading | Total DPV | Average dose | T_{50%} |
|---|---|---|---|---|
| ARV | (mg) (wt %) | released (mg) | (μg/day) | (days) |
| RAL | 8.37 (15.7) | 1.30 | 46.31 | 101 |
| MVC | 9.00 (17.0) | 2.18 | 77.75 | 58 |
| ETR | 10.94 (19.5) | 0.43 | 15.24 | 300 |

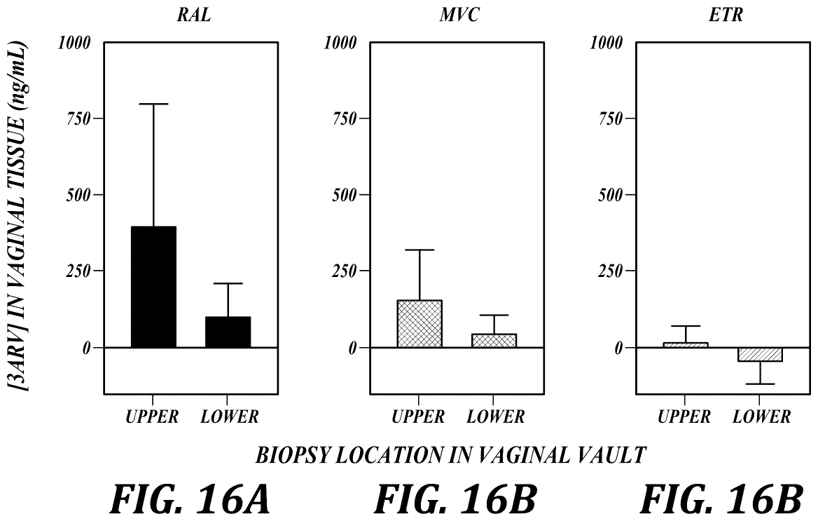

FIGS. 16A-16C are graphs showing concentrations of anti-retroviral pharmaceutical compositions in the vaginal vault, in accordance with the present technology. On the vertical axis are the three ARVs (RAL, MVC, ETR) in vaginal tissue in ng/ml. On the horizontal axis is the upper and lower vaginal vault samples (i.e., the location in the vaginal vault). As shown, the concentration of the ARVs follow a uterine distance-dependent gradient.

### Example 5: Study of Matrix Delivery System Including Pritelivir (PTV)

A monolithic or “matrix” delivery system was also tested loaded with pritelivir (PTV). An example illustration of the intrauterine system used in the study is shown in FIG. 1C. The intrauterine device was tested in vitro for 480 hours.

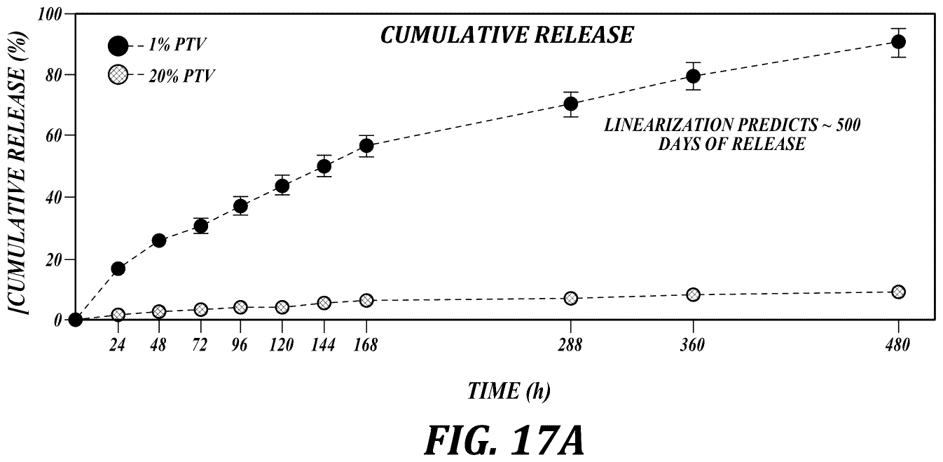

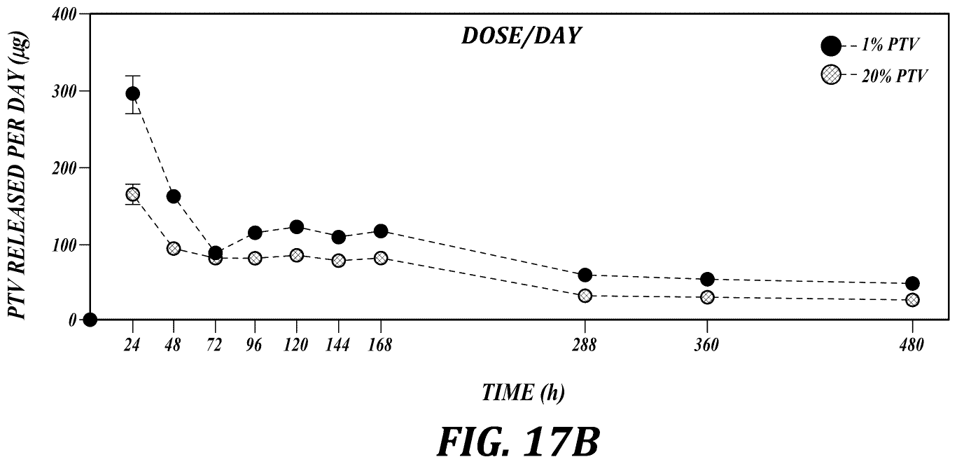

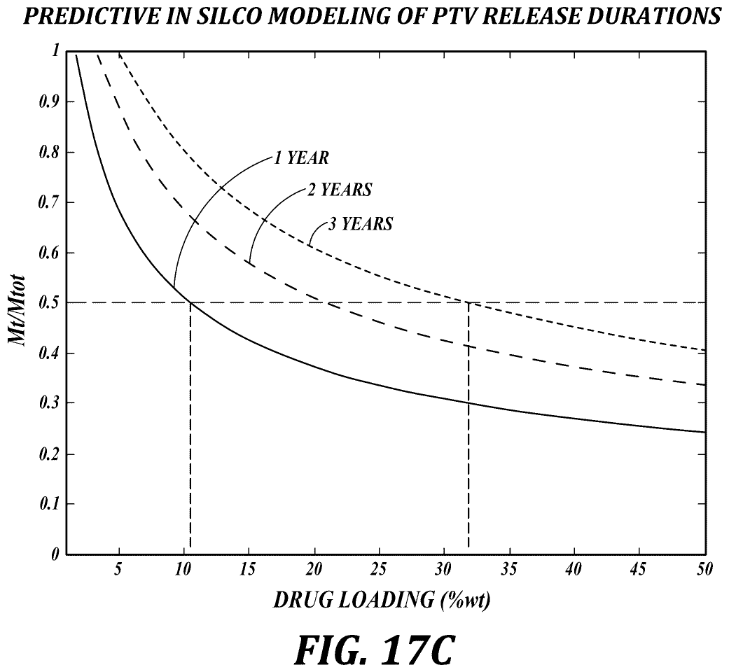

FIGS. 17A-17C are graphs showing the in vitro release of pritelivir (PTV), in accordance with the present technology. FIG. 17A shows the cumulative release of PTV over time. On the vertical axis is cumulative release in percentage. FIG. 17B shows the PTV released per day in micrograms. On the horizontal axes of both FIG. 17A-17B is the time in hours. The PTV device was loaded with 20-30 mg of PTV, and the duration of use of the device is about 3-4 years.

### Example 6: Study of Intrauterine Devices Loaded with Acyclovir or Doxycycline for HSV and Chlamydia Treatment, Respectively

A delivery system was loaded with acyclovir (ACV). High saturation matrixes were able to consistently release at least 56 micrograms per day of ACV, with a minimum of 109 micrograms per day and a maximum of 1112 micrograms per day.

A delivery system was also loaded with doxycycline. The IC₅₀ threshold of 0.497 micrograms per mL was successfully met by both low saturation and high saturation devices loaded with doxycycline.

The data for both ACV and doxycycline was also fit to the Higuchi model of drug release as described herein. The average cumulative fraction of release for each experimental group was compared with square root of time. A line of best fit was calculated using simple linear regression.

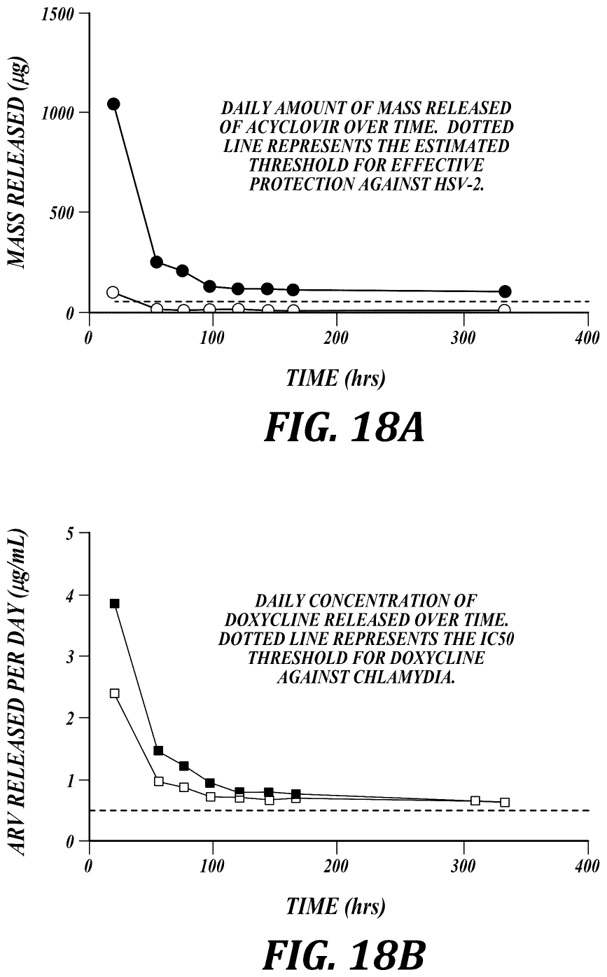

FIGS. 18A-18B are graphs showing daily doses of released acyclovir and doxycycline, respectively, in accordance with the present technology. FIG. 18A shows the daily amount of mass released of acyclovir over time. The dotted line represents the estimated threshold for effective protection against HSV-2. FIG. 18B shows the daily concentration of doxycycline released over time. The dotted line represents the IC50 threshold for doxycycline against chlamydia.

### Example 7: Screening Ethylene-Vinyl Acetate Polymers for Release of 1% Dapivirine

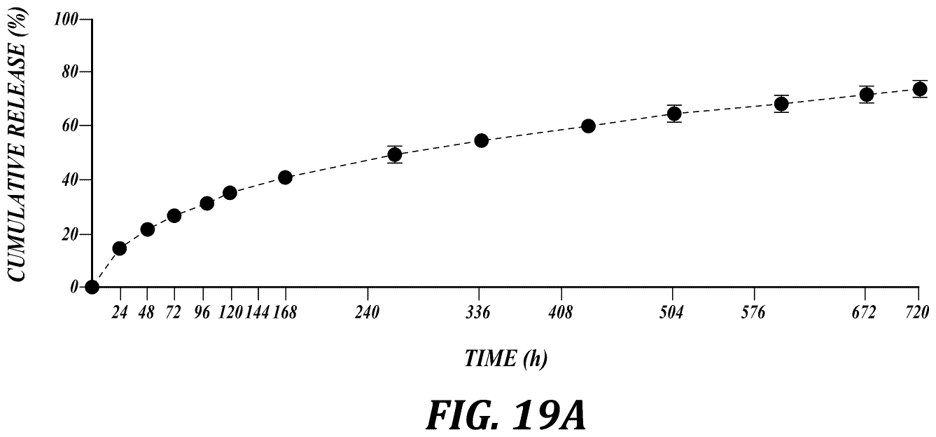

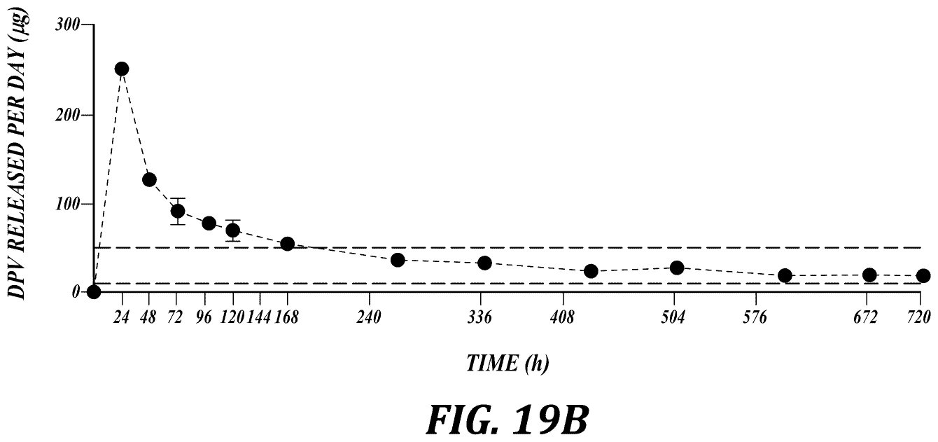

Ethylene-vinyl acetate polymers (EVA) were also investigated for the use in the intrauterine systems described herein. FIGS. 19A-19B are graphs showing release for dapirivine (DPV) in ethylene-vinyl acetate polymers (EVAs), in accordance with the present technology.

FIG. 19A shows the cumulative release in percentage of DPV over time in hours. As shown, about 80% of the DPV loaded in the delivery system was released within 720 hours.

FIG. 19B shows the DPV release per day in micrograms over time in hours. The daily DPV release ranged from about 250 micrograms per day to less than 50 micrograms per day.

Accordingly, EVAs may be utilized as the core polymer for delivery systems described herein.

In some embodiments, disclosed herein is a drug delivery system configured to be retained in a uterus of a patient, and to deliver one or more active compounds, wherein the one or more active compounds have non-systemic extrauterine biological targets.

In some embodiments, the non-systemic extrauterine biological targets are in a lower female reproductive tract (LFGT) of the patient. In some embodiments, the non-systemic extrauterine biological targets are in an anus or a rectum of the patient. In some embodiments, the delivery system is configured to accumulate the one or more active compounds in the LFGT, the anus, or the rectum for an extended duration. In some embodiments, the intrauterine system is configured to facilitate uterine-to-LFGT transport of the one or more active compounds, and wherein the one or more active compounds are detectable in tissue, local secretions, or a combination thereof at least 7 days from retention of the intrauterine system into a uterus.

In some embodiments, the tissue is selected from cervical tissue, uterus tissue, endometrium tissue, fallopian tube tissue, vaginal tissue, vulvar tissue, vestibule tissue, vestibular glands, vulva tissue, or a combination thereof. In some embodiments, the one or more active compounds have a steady-state drug concentration of at least 0.5-25 ng/mg in the tissue. In some embodiments, the one or more active compounds have a steady-state drug concentration of at least 0.5-25 ng/mL in the local secretions.

In some embodiments, the delivery system includes a core formed from a core polymer, wherein the one or more active compounds are dispersed within the polymer core.

In some embodiments, the core polymer comprises thermoplastic polyurethane (TPU). In some embodiments, the core polymer is hydrophobic. In some embodiments, the delivery system further comprises a sheath membrane surrounding the core. In some embodiments, the sheath membrane has a thickness between 50-500 μm. In some embodiments, the sheath membrane is formed from a sheath polymer. In some embodiments, the sheath polymer is different than the core polymer. In some embodiments, the sheath polymer is hydrophobic. In some embodiments, the sheath polymer comprises TPU.

In some embodiments, the delivery system further includes an opening. In some embodiments, an intrauterine frame is configured to thread through the opening.

In some embodiments, the one or more active compounds are configured to treat one or more sexually transmitted infections. In some embodiments, the one or more sexually transmitted infections are selected from human immunodeficiency virus (HIV), herpes, chlamydia, gonorrhea, syphilis, and bacterial vaginosis. In some embodiments, the one or more active compounds is a pharmaceutical composition. In some embodiments, the pharmaceutical composition is an anti-infective pharmaceutical composition. In some embodiments, the anti-infective pharmaceutical composition is an antiviral pharmaceutical composition. In some embodiments, the anti-infective pharmaceutical composition is an anti-retroviral (ARV) pharmaceutical composition selected from raltegravir (RAL), maraviroc (MVC), etravirine (ETR), dapivirine (DPV), or a combination thereof. In some embodiments, the anti-infective pharmaceutical composition is an antiviral pharmaceutical composition configured to inhibit Herpes Simplex Virus (HSV) selected from helicase primase inhibitors nucleoside analogs. In some embodiments, the anti-infective pharmaceutical composition is an antiviral pharmaceutical composition selected from pritelivir, acyclovir, or a combination thereof.

In some embodiments, the pharmaceutical composition accumulates non-systemically in cervical tissues, vaginal tissues, vaginal vestibule tissues, rectum tissues, vaginal fluids, or a combination thereof in a concentration that is uterine-distance dependent. In some embodiments, the pharmaceutical composition is released from the delivery system at an effective level for up to about ten years. In some embodiments, the effective level ranges from about 1 μg to about 500 μg a day.

In another aspect, disclosed herein is at least one drug delivery system as described herein, and an intrauterine frame.

In some embodiments, the intrauterine system is further configured to prevent pregnancy.

In some embodiments, the intrauterine system further comprises one or more hormonal elements, non-hormonal elements, or a combination thereof.

In some embodiments, the drug delivery system is a first drug delivery system, and wherein the intrauterine system comprises a plurality of drug delivery systems. In some embodiments, each drug delivery system includes a polymer core, wherein the one or more active compounds are dispersed within the polymer core. In some embodiments, each drug delivery system further comprises a sheath membrane surrounding the polymer core. In some embodiments, each drug delivery system releases a different active compound of the one or more active compounds. In some embodiments, the one or more active compounds are an anticancer drug.

In yet another aspect, disclosed herein is a method of using the drug delivery system disclosed herein, the method including retaining the drug delivery system in a uterus, and delivering the one or more active compounds to non-systemic extrauterine biological targets in the LFGT, the anus, or the rectum.

In some embodiments, delivering the one or more active compounds further includes accumulating the one or more active compounds in the LFGT, the anus, or rectum for at least one year. In some embodiments, delivering the one or more active compounds further includes accumulating a steady-state drug concentration of the one or more active compounds of at least 0.5 to 25 ng/mg in tissue, a steady-state drug concentration of the one or more active compounds of at least 0.5 to 25 ng/mL in local secretions, or a combination thereof.

In some embodiments, method further includes treating one or more sexually transmitted infections with the one or more active compounds.

While illustrative embodiments have been illustrated and described, it will be appreciated that various changes can be made therein without departing from the spirit and scope of the invention.

The complete disclosure of all patents, patent applications, and publications, and electronically available material cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims. The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure.

Specific elements of any foregoing embodiments can be combined or substituted for elements in other embodiments. Moreover, the inclusion of specific elements in at least some of these embodiments may be optional, wherein further embodiments may include one or more embodiments that specifically exclude one or more of these specific elements. Furthermore, while advantages associated with certain embodiments of the disclosure have been described in the context of these embodiments, other embodiments May also exhibit such advantages, and not all embodiments need necessarily exhibit such advantages to fall within the scope of the disclosure.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure.

At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

All of the references cited herein are incorporated by reference. Aspects of the disclosure can be modified, if necessary, to employ the systems, functions, and concepts of the above references and application to provide yet further embodiments of the disclosure. These and other changes can be made to the disclosure in light of the detailed description.

It will be appreciated that, although specific embodiments of the disclosure have been described herein for purposes of illustration, various modifications may be made without deviating from the spirit and scope of the disclosure. Accordingly, the disclosure is not limited except as by the claims. While illustrative embodiments have been illustrated and described, it will be appreciated that various changes can be made therein without departing from the spirit and scope of the disclosure.

We claim:

1. A drug delivery system configured to be retained in a uterus of a patient, and to deliver one or more active compounds, wherein the one or more active compounds have non-systemic extrauterine biological targets.

2. The drug delivery system of claim 1, wherein the non-systemic extrauterine biological targets are in a lower female genital tract (LFGT) of the patient.

3. The drug delivery system of claim 1, wherein the non-systemic extrauterine biological targets are in an anus or a rectum of the patient.

4. The drug delivery system of claim 1, wherein the delivery system is configured to accumulate the one or more active compounds in the LFGT, the anus, or the rectum for an extended duration.

5. The drug delivery system of claim 1, wherein the intrauterine system is configured to facilitate uterine-to-LFGT transport of the one or more active compounds, and wherein the one or more active compounds are detectable in tissue, local secretions, or a combination thereof at least 7 days from placement of the intrauterine system into a uterus.

6. The drug delivery system of claim 5, wherein the tissue is selected from cervical tissue, endometrium tissue, vaginal tissue, vulvar tissue, vestibule tissue, vestibular glands, or a combination thereof.

7. The drug delivery system of claim 5, wherein the one or more active compounds have a steady-state drug concentration of at least 0.5-25 ng/mg in the tissue.

8. The drug delivery system of claim 5, wherein the one or more active compounds have a steady-state drug concentration of at least 0.5-25 ng/ml in the local secretions.

9. The drug delivery system of claim 1, wherein the delivery system comprises:
a core formed from a core polymer, wherein the one or more active compounds are dispersed within the polymer core.

10. The drug delivery system of claim 9, wherein the core polymer comprises thermoplastic polyurethane (TPU).

11. The drug delivery system of claim 9, wherein the delivery system further comprises a sheath membrane surrounding the core.

12. The drug delivery system of claim 1, wherein the one or more active compounds are configured to prevent, treat, or a combination thereof, one or more conditions selected from human immunodeficiency virus (HIV), chlamydia, gonorrhea, syphilis, Herpes Simplex Virus (HSV), and bacterial vaginosis.

13. The drug delivery of claim 1, wherein the one or more active compounds are selected from raltegravir (RAL), maraviroc (MVC), etravirine (ETR), dapivirine (DPV), pritelivir, acyclovir, doxycycline, or a combination thereof.

14. The drug delivery system of claim 1, wherein the one or more active compounds are configured to accumulate non-systemically in cervical tissues, vaginal tissues, vaginal vestibule tissues, rectum tissues, vaginal fluids, or a combination thereof in a concentration that is uterine-distance dependent.

15. The drug delivery system of claim 14, wherein the one or more compounds is configured to release from the delivery system at an effective level for up to about ten years.

16. The drug delivery system of claim 15, wherein the effective level ranges from about 1 μg to about 500 μg a day.

17. An intrauterine system, comprising:
at least one drug delivery system according to claim 1, and
an intrauterine frame.

18. The intrauterine system of claim 17, wherein the intrauterine system is further configured to prevent pregnancy.

19. The intrauterine system of claim 17, wherein the drug delivery system is a first drug delivery system, and wherein the intrauterine system further comprises a second drug delivery system, wherein the first drug delivery system releases an active compound of the one or more active compounds and wherein the second drug delivery system releases a different active compound of the one or more active compounds.

20. A method of using the drug delivery system of claim 1, the method comprising:
retaining the drug delivery system in a uterus; and
delivering the one or more active compounds to non-systemic extrauterine biological targets in the LFGT, the anus, or the rectum.
